(19) United States
(12) Patent Application Publication
(10) Pub. No.: US 2016/0237498 A1
(43) Pub. Date: Aug. 18, 2016

(54) PREDICTION AND TREATMENT OF HEART FAILURE

(71) Applicant: The Regents of The University of Colorado, a body corporate, Denver, CO (US)

(72) Inventors: Michael BRISTOW, Aurora, CO (US); Carmen SUCHAROV, Aurora, CO (US); Matthew TAYLOR, Denver, CO (US); Dobromir SLAVOV, Aurora, CO (US)

(73) Assignee: The Regents of the University of Colorado, a body corporate, Denver, CO (US)

(21) Appl. No.: 15/028,117

(22) PCT Filed: Oct. 3, 2014
(86) PCT No.: PCT/US14/58962

§ 371 Date: Apr. 8, 2016

Related U.S. Application Data
(60) Provisional application No. 61/888,845, filed on Oct. 9, 2013.

(51) Int. Cl. C12Q 1/68

(52) U.S. Cl. CPC C12Q 1/6883; C12Q 2600/156

(57) ABSTRACT

Chronic activation of the β-Adrenergic Receptor (β-AR) can have deleterious effects on the heart, and animal models over-expressing the β-AR develop heart failure. In the classical β-AR pathway, activation of the receptor results in increased cyclic AMP(cAMP) levels. However, β-ARs are desensitized in the failing heart and cAMP levels are decreased. Phosphodiesterase 3A (PDE3A) hydrolyzes cAMP in certain subcellular compartments in cardiac myocytes, regulating cAMP levels and subsequent protein kinase A mediated cell signaling. By virtue of being freely diffusable intracellularly and being reduced in failing myocardial tissue, cAMP is reduced in certain important cardiac myocyte subcellular compartments such as the microdomain occupied by phospholamban.

This application claims benefit of priority to U.S. Provisional Application Ser. No. 61/888,845, filed Oct. 9, 2013, the entire contents of which are hereby incorporated in their entirety.

This invention was made with government support under grant nos. NIH 2R01 HL48013, NIH K01 HL088708, and R21 HL097123 awarded by the National Institutes Health. The government has certain rights in the invention.

### BACKGROUND OF THE INVENTION

I. Field of the Disclosure

Embodiments of this disclosure are directed generally to biology, molecular genetics, and medicine. Certain embodiments are directed to identifying a subject that would benefit from phosphodiesterase inhibitor therapy.

II. Background

Activation of β-adrenergic receptors (β-ARs) increases adenylyl cyclase (AC) activity, resulting in cAMP synthesis, activation of protein kinase A (PKA) and phosphorylation of various downstream effectors. The cardiac β-AR/AC/cAMP/PKA axis has an important role in stimulating heart rate and myocardial contraction and relaxation. In the failing human heart β-ARs are desensitized, and cAMP levels are decreased despite increased adrenergic activity. The desensitization of β-AR signal transduction mechanisms may compromise cardiac function as well as redirect signaling to more biologically adverse “PKA-independent” pathways, such as Ca²⁺/calmodulin-dependent protein kinase II (CaMKII) (Bristow, 2011).

Cyclic nucleotide phosphodiesterases (PDEs) hydrolyze the phosphodiester bond in cyclic adenosine or guanosine monophosphate (cAMP or cGMP) for conversion to their respective 5′ monophosphates (Movesian et al., 2009). In the failing heart an increase in cAMP to normal or elevated levels could potentially be achieved by inhibition of PDEs (Feldman et al., 1987). However, several clinical trials have shown increased mortality (Packer et al., 1991; Cohn et al., 1998) or lack of efficacy (Metra et al., 2009) in adult heart failure (HF) patients treated with PDE3 inhibitors. The increase in mortality appears to be due to pro-arrhythmic effects interacting with other clinical variables to produce an increase in sudden death (Holmes et al., 1985; Teerlink et al., 2000), while the lack of effectiveness when a PDE3 inhibitor was used with a β-blocker appeared to be due in part to response heterogeneity (Metra et al., 2009). In adult HF populations, PDE3 inhibitors confer short-term hemodynamic benefits, but tolerance, in the form of decreased levels of cAMP, may develop when treatment is extended (Maisel et al., 1989).

Effects of cAMP are classically attributed to the phosphorylation of proteins that affect excitation/contraction coupling, including L-Type Ca²⁺ channels (LTCCs), the sarcoplasmic reticulum ATPase 2 (SERCA2) regulatory protein phospholamban (PLN), ryanodine receptor 2 (RyR2), phosphatase 1 inhibitor and various contractile proteins (Mika et al., 2012). In order to regulate localized cAMP-mediated signaling, PDEs are compartmentalized intracellularly (Bristow, 2011; Mika et al., 2012), and PDE3A is present in a microdomain with SERCA2 and PLN (Beca et al., 2013). Three isoforms of PDE3A have been identified in human myocardium, resulting from alternative transcriptional or translational start sites (Wechsler et al., 2002). PDE3A1, the longest isoform, is localized to microsomal fractions, while two shorter isoforms, PDE3A2 and PDE3A3, are present in microsomal and cytosolic fractions (Wechsler et al., 2002).

The inventors have also shown that a 29-nucleotide insertion (INS) or deletion (DEL) polymorphism is present in the PDE3A promoter, which regulates transcription via a downstream cAMP response element (CRE). In HF patients, treatment with PDE3 inhibitors results in increased PDE3A1 mRNA expression and catalytic activity in DEL homozygotes but not in INS homozygotes. These results suggest that PDE3 inhibitor treatment may be more efficacious in INS homozygote patients, where tolerance resulting from up-regulation of PDE3A gene expression would not be present (U.S. Patent Publication 2013/0203827).

### SUMMARY OF THE INVENTION

Chronic activation of the β-Adrenergic Receptor (β-AR) can have deleterious effects on the heart, and animal models over-expressing the β-AR develop heart failure. In the classical β-AR pathway, activation of the receptor results in increased cyclic AMP (cAMP) levels. However, β-ARs are desensitized in the failing heart and cAMP levels are decreased. Phosphodiesterase 3A (PDE3A) hydrolyzes cAMP in certain subcellular compartments in cardiac myocytes, regulating cAMP levels and subsequent protein kinase A mediated cell signaling. By virtue of being freely diffusable intracellularly and being reduced in failing myocardial tissue, cAMP is reduced in certain important cardiac myocyte subcellular compartments such as the microdomain occupied by phospholamban and SR Ca²⁺ ATPase. This results in decreased protein kinase A mediated phosphorylation of phospholamban, which increases the inhibitory effects of this regulatory protein on SR Ca²⁺ ATPase and decreases contractile function. It has been postulated that PDE3A inhibition will benefit the heart failure clinical syndrome by restoring cAMP levels in the phospholamban microdomain/subcellular compartment. However, a recently published large clinical trial showed benefits of a PDE3 inhibitor on only a subset of patients.

Thus, in accordance with the present disclosure, there is provided a method of prophylactically treating heart failure comprising treating a subject after the subject is determined to exhibit a −1130 to −1159 deletion polymorphism in the promoter of a phosphodiesterase type 3A (PDE3A) gene, wherein said treating does not comprise a PDE3A inhibitor. The treatment may comprise a beta blocker or an agent that prevents reductions in intracellular cAMP. The subject may be heterozygous for the deletion polymorphism or homozygous for the deletion polymorphism.

Determining may comprise nucleic acid amplification, nucleic acid hybridization, restriction fragment length polymorphism (RFLP) analysis, single stranded conformational polymorphism (SSCP) analysis, nucleic acid sequencing, denaturing high performance liquid chromatography, allele specific amplification, allele specific hybridization, comparative genome hybridization, and/or Southern blotting. Nucleic acid amplification may comprise polymerase chain reaction amplification or ligase chain reaction amplification. Nucleic acid hybridization detection method may comprise an allele specific oligonucleotide probe or a microarray of nucleic acid probes. The subject may have been diagnosed with one or more of hypertension, myocardial infarction, atherosclerosis, cardiac hypertrophy, left ventricular remodeling, or coronary artery disease. The subject is a carrier of a gene known to cause cardiomyopathy.

In another embodiment, there is provided a method for predicting development of heart failure in a subject not suffering from heart failure comprising analyzing a biological sample from the subject for the presence of a −1130 to −1159 insertion/deletion polymorphism in the promoter of one or more phosphodiesterase type 3A (PDE3A) genes of the subject, wherein a subject determined to be homozygous or heterozygous for the deletion polymorphism is at increased risk of developing heart failure as compared to an individual who is homozygous for the insertion polymorphism. The subject may be heterozygous for the deletion polymorphism or homozygous for the deletion polymorphism.

Determining may comprise nucleic acid amplification, nucleic acid hybridization, restriction fragment length polymorphism (RFLP) analysis, single stranded conformational polymorphism (SSCP) analysis, nucleic acid sequencing, denaturing high performance liquid chromatography, allele specific amplification, allele specific hybridization, comparative genome hybridization, and/or Southern blotting. Nucleic acid amplification may comprise polymerase chain reaction amplification or ligase chain reaction amplification. Nucleic acid hybridization detection method may comprise an allele specific oligonucleotide probe or a microarray of nucleic acid probes. The subject may have been diagnosed with one or more of hypertension, myocardial infarction, atherosclerosis, cardiac hypertrophy, left ventricular remodeling, or coronary artery disease. The subject is a carrier of a gene known to cause cardiomyopathy. The method may further comprise scheduling said subject for one or more follow up diagnostic procedures selected from measurement of ventricular volume, measurement of ejection fraction, and/or surveillance for progressive remodeling. Such diagnostics may be scheduled on a more frequent basis even if such would have already been indicated.

In yet another embodiment, there is provided a method for treating early stage heart failure in a subject comprising treating a subject after the subject is determined to exhibit a −1130 to −1159 deletion polymorphism in the promoter of phosphodiesterase type 3A (PDE3A) gene, wherein said treating does not comprise a PDE3A inhibitor. The treatment may comprise a beta blocker or an agent that prevents reductions in intracellular cAMP. The subject may be heterozygous for the deletion polymorphism or homozygous for the deletion polymorphism.

Determining may comprise nucleic acid amplification, nucleic acid hybridization, restriction fragment length polymorphism (RFLP) analysis, single stranded conformational polymorphism (SSCP) analysis, nucleic acid sequencing, denaturing high performance liquid chromatography, allele specific amplification, allele specific hybridization, comparative genome hybridization, and/or Southern blotting. Nucleic acid amplification may comprise polymerase chain reaction amplification or ligase chain reaction amplification. Nucleic acid hybridization detection method may comprise an allele specific oligonucleotide probe or a microarray of nucleic acid probes. The subject may have been diagnosed with one or more of hypertension, myocardial infarction, atherosclerosis, cardiac hypertrophy, left ventricular remodeling, or coronary artery disease. The subject is a carrier of a gene known to cause cardiomyopathy.

In certain aspects the methods described herein can further comprise obtaining a biological sample from the subject. A biological sample can be a blood sample, a buccal smear, a tissue sample, or a primary culture of somatic cells from the subject. In certain aspects analyzing the sample comprises performing nucleic acid sequencing, restriction digestion, allele-specific nucleic acid amplification, single-stranded conformational polymorphism analysis, or allele specific hybridization analysis. The methods described herein can further comprising preparing a report containing information regarding the genotype of one or more PDE3A genes of the subject. In a further aspect the subject has symptoms of or has been diagnosed with heart failure.

Embodiments are directed to methods comprising detecting a nucleic acid deletion can comprise at least one allele specific amplification primer or allele specific nucleic acid probe. The nucleic acid insertion can be detected using nucleic acid amplification, nucleic acid hybridization, restriction fragment length polymorphism (RFLP) analysis, single stranded conformational polymorphism (SSCP) analysis, nucleic acid sequencing, denaturing high performance liquid chromatography, comparative genome hybridization, and/or Southern blotting. In certain aspects nucleic acid amplification comprises polymerase chain reaction amplification or ligase chain reaction amplification. In a further aspect nucleic acid hybridization detection method comprises an allele specific oligonucleotide probe or a microarray of nucleic acid probes. Another aspect is a kit for genotyping a phosphodiesterase type 3A gene.

To achieve these methods, a doctor, medical practitioner, or their staff may obtain directly from the subject a biological sample for evaluation. The sample may be analyzed by the practitioner or their staff, or it may be sent to an outside or independent laboratory. The medical practitioner may be cognizant of whether the test is providing information regarding the subject's PDE3A genes, or the medical practitioner may be aware only that the test indicates directly or indirectly that the genotype of the patient reflects the deletion polymorphism phenotype.

Similarly, the medical practitioner may be cognizant of whether the test is providing information regarding the patient's PDE3A genes or the medical practitioner may be aware only that the test indicates directly or indirectly that the genotype of the patient reflects the homozygous or the heterozygous deletion genotype.

The term “genotyping” is refers to the physical manipulation and transformation of a biological sample to determine genotype information contained in the sample. Obtaining genotype information refers to obtaining some or all of the results of a genotyped sample.

Certain embodiments are directed to a tangible, computer-readable medium comprising a genotype of a subject, wherein the genotype exhibits the presence or absence of deletion in the PDE3A gene. In certain aspects the medium comprising the genotype of the subject exhibits the presence of a deletion in the promoter of one or more PDE3A gene. Moreover, in further embodiments the medium or method involves an algorithm that determines the risk of a phenotype based on the genotype information. A report may be subsequently prepared with the results of the genotyping and/or predicted phenotype.

Moreover, in some methods a clinician may or may not order a genotyping test for a patient. In other embodiments, a clinician may retrieve a biological sample from a patient, which may or may not constitute a biopsy. In further embodiments, methods may involve receiving genotype information about a patient's sample and subsequently treating a patient based on the genotype information. In further methods, an algorithm may be run to evaluate possible phenotype based on genotyping information.

The term “polymorphism,” as used herein, refers to a difference in the nucleotide or amino acid sequence of a given nucleotide or amino acid region as compared to a nucleotide or amino acid sequence in the corresponding region of another individual of the same species. In particular, the species is human. A polymorphism is generally defined in relation to a “reference” sequence. In the subject application, “reference” sequence and “wild type” sequence are used interchangeably. Nucleotide polymorphisms include single nucleotide differences, differences in sequence of more than one nucleotide, and single or multiple nucleotide insertions, inversions, substitutions, and deletions Amino acid polymorphisms include single amino acid differences, differences in sequence of more than one amino acid, and single or multiple amino acid insertions, substitutions, and deletions.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides. The term biological sample encompasses a clinical sample, and also includes cells in culture, cell supernatants, cell lysates, serum, plasma, biological fluid, and tissue samples. In one embodiment, the sample is collected by the individual. For example, an individual can collect a swap of tissue from the inside of the cheek for use as a nucleic acid sample. As known in the art, many types of samples can be used for the extraction of nucleic acids.

The term “therapeutically effective amount” means an amount that is effective in treating a particular disorder; that is an amount that is effective for reducing the severity of one or more symptoms associated with the particular disorder for which treatment is sought. The term “ameliorate,” as used for instance in the amelioration of a particular condition means to make one or more symptoms of the condition at least more tolerable, if not better. The term ameliorate does not necessarily mean an increase in toleration of all symptoms associated with a disorder and does not necessarily mean a complete reduction in the severity of one or more symptoms associated with a disorder.

As used herein, the term “heart failure” is broadly used to mean any condition that reduces the ability of the heart to pump blood. As a result, congestion and edema develop in the tissues. Most frequently, heart failure is caused by decreased contractility of the myocardium, resulting from reduced coronary blood flow; however, many other factors may result in heart failure, including damage to the heart valves, vitamin deficiency, and primary cardiac muscle disease. Though the precise physiological mechanisms of heart failure are not entirely understood, heart failure is generally believed to involve disorders in several cardiac autonomic properties, including sympathetic, parasympathetic, and baroreceptor responses. The phrase “manifestations of heart failure” is used broadly to encompass all of the sequelae associated with heart failure, such as shortness of breath, pitting edema, an enlarged tender liver, engorged neck veins, pulmonary rates and the like including laboratory findings associated with heart failure.

The term “treatment” or equivalents encompasses the improvement and/or reversal of the symptoms of heart failure (i.e., the ability of the heart to pump blood). “Improvement in the physiologic function” of the heart may be assessed using any of the measurements described herein (e.g., measurement of ejection fraction, fractional shortening, left ventricular internal dimension, heart rate, etc.), as well as any effect upon subject's survival. In certain embodiments, a patient administers the drug to himself/herself.

The term “dilated cardiomyopathy” refers to a type of heart failure characterized by the presence of a symmetrically dilated left ventricle with poor systolic contractile function and, in addition, frequently involves the right ventricle.

As used herein, the term “cardiac hypertrophy” refers to the process in which adult cardiac myocytes respond to stress through hypertrophic growth. Such growth is characterized by cell size increases without cell division, assembling of additional sarcomeres within the cell to attempt to increase force generation, and an activation of a fetal cardiac gene program that inherently reduces myocardial function. Pathologic cardiac hypertrophy is associated with increased risk of morbidity and mortality and ultimately to decreased contractile function, and thus studies aimed at understanding the molecular mechanisms of cardiac hypertrophy could have a significant impact on human health.

As used herein, the terms “antagonist” and “inhibitor” refer to molecules, compounds, or nucleic acids which inhibit the action of a protein. Antagonists and inhibitors may include proteins, nucleic acids, carbohydrates, or any other molecules which bind or interact with a protein of interest.

The term “phosphodiesterase inhibitor” or “PDE inhibitor” refers to a chemical compound or entity that is capable of blocking, either partially or completely, the activity of a phosphodiesterase enzyme. Some PDE inhibitors exhibit a degree of specificity for one PDE subtype (e.g., phosphodiesterase type 3 (PDE3)). The term “PDE3 inhibitor” refers to chemical compounds that are selective for phosphodiesterase type 3, which includes inhibition of the PDE3A and PDE3B isoforms. The use of derivatives of known PDE3 inhibitors is encompassed by the methods of the present disclosure. Indeed any compound, which functionally inhibits PDE3A is encompassed by the methods of the present disclosure.

As used herein, the term “genotype” refers to the actual genetic make-up of an organism, while “phenotype” refers to physical traits displayed by an individual.

Other embodiments of the disclosure are discussed throughout this application. Any embodiment discussed with respect to one aspect of the disclosure applies to other aspects of the disclosure as well and vice versa. The embodiments in the Example section are understood to be embodiments of the disclosure that are applicable to all aspects of the disclosure.

The terms “inhibiting,” “reducing,” or “prevention,” or any variation of these terms, when used in the claims and/or the specification includes any measurable decrease or complete inhibition to achieve a desired result.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any method or composition of the disclosure, and vice versa. Furthermore, compositions and kits of the disclosure can be used to achieve methods of the disclosure.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.” It is also contemplated that anything listed using the term “or” may also be specifically excluded.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any foam of containing, such as “contains” and “contain”) are inclusive or open-ended and do not exclude additional, unrecited elements or method steps.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1. Identification of a polymorphic region on the PDE3A promoter. An insertion of 29 nucleotides (nt) was identified 1130 bp upstream from the PDE3A transcription start site. The 29 nt sequence is shown (SEQ ID NO:1). A putative cAMP response element (CRE) is present 1069 bp upstream from the PDE3A transcription start site.

FIGS. 2A-B. Deletion polymorphism results in increased cAMP-dependent promoter activity. (FIG. 2A) NRVMs were transfected with DEL or INS promoter constructs. 24 hours after transfection cells were treated with 1 μmol/L dibutyryl cAMP or 5 μmol/L enoximone for 48 hours. (FIG. 2B) NRVMs were transfected with the DEL promoter construct+/−a CRE binding site. 24 hours after transfection cells were treated with cAMP or enoximone for 48 hours. ANOVA/Scheffe test p values are shown. N=8 different transfections, 4-6 wells/transfection.

FIGS. 3A-B. PDE3A1 mRNA abundance analyzed by RT-PCR in non-failing and failing LVs, by −1130 INS/DEL genotype. (FIG. 3A) All genotypes. (FIG. 3B) Failing hearts only, INS homozygotes vs. heterozygotes or DEL homozygotes (DEL carriers). Values are normalized to 18S rRNA and then to Non-failing Ins/Ins as 100%, represented as 1.0 on the y axis. P value is by t-test.

FIG. 4. PDE3A1 mRNA abundance analyzed by RT-PCR in non-failing and failing LVs with and without PDE3 inhibitor treatment, by INS/DEL genotype reveals increased expression in DEL homozygous failing LVs with previous PDE inhibitor treatment. Normalization methods are described in FIGS. 3A-B. P values are by ANOVA/Scheffe test.

FIGS. 5A-D. PDE enzyme activity in non-failing and failing human LVs (LVs). (FIG. 5A) PDE3 activity in non-failing vs. all failing hearts, by INS/DEL genotype; p value for INS/DEL is by t-test. (FIG. 5B) PDE3 activity in INS homozygotes vs. heterozygotes+DEL homozygotes (DEL carriers), p value by t-test. (FIG. 5C) PDE3 activity by genotype in non-failing and failing hearts separated by presence or absence of PDE3 inhibitor treatment, showing increased activity in PDE inhibitor treated failing LVs in heterozygotes and DEL homozygotes; p values by ANOVA/Scheffe test. (FIG. 5D) PDE3 activity in failing hearts treated with PDE3 inhibitors, showing DEL allele dose related increased activity; p values by ANOVA/Scheffe test.

FIGS. 6A-B. Tissue homogenate cAMP levels in non-failing and failing human hearts, by INS/DEL genotype. (FIG. 6A) Non-failing and all failing hearts. (FIG. 6B) Failing hearts from patients treated with PDE3 inhibitors. P values by ANOVA/Scheffe test.

FIG. 7. Schematic representation of the results presented in FIGS. 1-6B. A 29-nucleotide insertion (INS) results prevents up-regulation of PDE3A1 mRNA levels by blocking activation of the CRE site in response to increase cAMP levels. DEL of that region results in up-regulation of PDE3 mRNA through derepression of the CRE site. PDE3 inhibition results in increased cAMP levels and PDE3A1 mRNA abundance in the DEL polymorphism, but increased PDE3A mRNA abundance is not observed in the INS polymorphism.

FIG. 8. Constructs for assessing position effects. The 29 nucleotide sequence was subcloned in different positions of the PDE3A promoter. The numbers indicate the position in relation to the transcription start site (+1).

FIG. 9. Positional effects for insertion region. NRVMs were transfected with the DEL construct, the wild-type INS, mutants and constructs where the position of the INS was altered. Firefly luciferase was normalized to Renilla luciferase. Bars represent the effect of cAMP treatment and are compared to each construct normalized to 100 (bar at 100). P-values are the result of the comparison of each construct to the wild-type INS (black bar). N=6-7 experiments. The order of the constructs from left to right in the graph is the same as from top to bottom in the legend on the right.

### DETAILED DESCRIPTION OF THE INVENTION

The present disclosure relates generally to assessing a PDE3A polymorphism in an individual and to predicting that individual's risk of progressing to heart failure. Alternatively, the present disclosure relates to determining the genotype for an individual at the PDE3A gene (e.g., determining if the subject has an insertion or deletion (indel) polymorphism) and further, providing an appropriate treatment, but that a therapy or continued and/or aggressive disease monitoring.

Pharmacogenomics allows a clinician or physician to target prophylactic or therapeutic treatments to individuals who will most benefit from the treatment and to avoid treatment of individuals who will experience symptomatic side effects. Thus, a physician or clinician may consider applying knowledge obtained in relevant pharmacogenomics studies in determining whether to (a) administer a certain treatment as well as tailoring the dosage, regimen, and/or therapeutically effective amounts to be administered so as to attain the effect desired by the treatment or (b) prescribe a particular diagnostic/monitoring regimen tailored to the risk of the individual of developing frank heart failure.

Heart failure is one of the leading causes of morbidity and mortality in the world. In the U.S. alone, estimates indicate that 3 million people are currently living with cardiomyopathy and another 400,000 are diagnosed on a yearly basis. Dilated cardiomyopathy (DCM), also referred to as “congestive cardiomyopathy,” is the most common form of the cardiomyopathies and has an estimated prevalence of nearly 40 per 100,000 individuals (Durand et al., 1995). Although there are other causes of DCM, familiar dilated cardiomyopathy has been indicated as representing approximately 20% of “idiopathic” DCM. Approximately half of the DCM cases are idiopathic, with the remainder being associated with known disease processes. For example, serious myocardial damage can result from certain drugs used in cancer chemotherapy (e.g., doxorubicin and daunoribucin).

In addition, many DCM patients are chronic alcoholics. Fortunately, for these patients, the progression of myocardial dysfunction may be stopped or reversed if alcohol consumption is reduced or stopped early in the course of disease. Peripartum cardiomyopathy is another idiopathic of DCM, as is disease associated with infectious sequelae. In sum, cardiomyopathies, including DCM, are significant public health problems. Additional methods of predicting heart failure, and hence for improving the patient outcome for individuals for which heart failure is a significant risk, are described.

### I. PHOSPHODIESTERASE (PDE)

cAMP signaling plays important roles in both physiologic and pathologic regulation of cardiac performance (Wang and Dhalla, 2000). cAMP is one of the most well characterized signaling molecules in β-AR signaling, but its contribution to Ang II signaling in cardiomyocytes is not fully understood. Clinical and experimental studies indicate that acute stimulation of β-AR/cAMP signaling increases myocyte contractility, which is beneficial. In contrast, chronic stimulation of cAMP signaling promotes myocyte apoptosis, which is harmful to the heart (Wang and Dhalla, 2000). The temporal and spatial features of cAMP production in the cell are controlled by adenylyl cyclases that catalyze the synthesis of cAMP, and phosphodiesterases (PDEs) that hydrolyze cAMP.

Phosphodiesterase as used herein refers to cyclic nucleotide phosphodiesterases. The cyclic nucleotide phosphodiesterases (PDE) comprise a group of enzymes that degrade the phosphodiester bond in the second messenger molecules cAMP and cGMP. They regulate the localization, duration, and amplitude of cyclic nucleotide signaling within subcellular domains. PDEs are therefore important regulators of signal transduction mediated by these second messenger molecules.

PDE enzymes are often targets for pharmacological inhibition due to their unique tissue distribution, structural properties, and functional properties Inhibitors of PDE can prolong or enhance the effects of physiological processes mediated by cAMP or cGMP by inhibition of their degradation by PDE. For example, Sildenafil (Viagra) is an inhibitor of cGMP specific phosphodiesterase type 5, which enhances the vasodilatory effects of cGMP in the corpus cavernosum and is used to treat erectile dysfunction. Cilostazol (Pletal) inhibits PDE3. This inhibition allows red blood cells to be more able to bend. This is useful in conditions such as intermittent claudication, as the cells can maneuver through constricted veins and arteries more easily.

PDE3 cyclic nucleotide phosphodiesterases bind cAMP and cGMP with high affinity and hydrolyze both substrates in a mutually competitive manner (Shaker et al., 2000). Two PDE3 genes have been discovered: PDE3A is expressed primarily in cardiac and vascular myocytes and platelets, whereas PDE3B is expressed primarily in adipocytes, hepatocytes, and pancreatic cells (Reinhardt et al., 1995). Phosphodiesterase type 3A (PDE3A) is an important regulator of cAMP-mediated responses within the cardiovascular system. PDE3 inhibitors that inhibit PDE3A (e.g. milrinone and enoximone) have inotropic effects attributable to the elevation of cAMP content in cardiac myocytes and vasodilatory effects attributable to the elevation of cAMP and/or cGMP content in vascular myocytes, and have been used to augment contractility and reduce afterload in patients with dilated cardiomyopathy (Movsesian, 1999). Although PDE3A is significantly down-regulated in one study in human failing hearts (Ding et al., 2005), in other studies in failing human hearts it was not decreased (Movsesian et al., 1991; Sucharov et al., 2008) Down-regulation of PDE3A expression/activity can induce apoptosis in cultured cardiomyocytes, due to PDE3A down-regulation-mediated induction of the proapoptotic transcriptional repressor ICER (Ding et al., 2005).

### II. DETECTION OF POLYMORPHISMS

The insertion/deletion polymorphism described herein is present in the promoter of PDE3A genes and affect the transcriptional regulation the PDE3A gene. The presence of the insertion polymorphism can be determined from the sequence of the PDE3A promoter or by using specific characteristics of the insertion polymorphism, e.g., amplicon size. As a result, a variety of different methodologies can be employed for the purpose of detecting polymorphisms in the promoter of PDE3A.

The insertion polymorphism comprises a binding site for the transcription factor Evi-1. Evi-1 (Ecotropic viral integration site-1) is a human protein encoded by the Evi-1 gene. The gene was first identified in AKXD murine myeloid tumors. Evi-1 is a nuclear transcription factor involved in many signaling pathways for both corepression and coactivation of various genes.

A. Nucleic Acids

Certain embodiments of the present disclosure concern various nucleic acids, including amplification primers, oligonucleotide probes, and other nucleic acid elements involved in the analysis of genomic DNA. In certain aspects, a nucleic acid comprises a wild-type, a mutant, or a polymorphic nucleic acid.

Based on the Human Genome Variation Society database the insertion/deletion polymorphism is annotated as rs145697127 and maps (based on the GRCh37.p13 Homo sapiens Primary Assembly and genome build 138) to a position on chromosome 12 identified as NC_000012.11:g.20520004_20520005 insTTCTCATATCTACTTATGTCATAATATTA (SEQ ID NO:1). This is equivalent to a position of −1130 (up to −1159 when inserted), counting upstream from the transcription start site of the PDE3A gene. As the insertion is too long for current automated population annotation algorithms no population frequency data are provided. Complementary annotation databases and programs currently exist and this same insertion/deletion also carries NCBI ‘submitted SNP’ annotations of: ss327415732, ss327508568, and ss552195051.

One of skill in the art knows how to design and use primers and probes for hybridization and amplification of the PDE3A promoter.

These definitions generally refer to a single-stranded molecule, but in specific embodiments will also encompass an additional strand that is partially, substantially or fully complementary to the single-stranded molecule. Thus, a nucleic acid may encompass a double-stranded molecule or a triple-stranded molecule that comprises one or more complementary strand(s) or “complement(s)” of a particular sequence comprising a molecule. As used herein, a single stranded nucleic acid may be denoted by the prefix “ss”, a double stranded nucleic acid by the prefix “ds”, and a triple stranded nucleic acid by the prefix “ts.”

1. Preparation of Nucleic Acids

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemical synthesis using phosphotriester, phosphite or phosphoramidite chemistry and solid phase techniques such as described in European Patent 266,032, incorporated herein by reference, or via deoxynucleoside H-phosphonate intermediates as described by Froehler et al., 1986 and U.S. Pat. No. 5,705,629, each incorporated herein by reference. In the methods of the present disclosure, one or more oligonucleotide may be used. In certain aspects amplification oligonucleotides can be designed on either side or overlapping with the boundaries of the insertion site. In a further aspect an oligonucleotide specific for insertion as compare to the PDE3A promoter lacking the insertional polymorphism can be designed. These oligonucleotides can varying in length from 5, 10, 15, 16, 17, 18, 19, 20, 21, 22, 23, 24, 25, 26, 27, 28, 29, 30, 50, nucleotides or more, including all values and ranges there between. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 2001, incorporated herein by reference).

2. Purification of Nucleic Acids

A nucleic acid may be purified on polyacrylamide gels, cesium chloride centrifugation gradients, chromatography columns or by any other means known to one of ordinary skill in the art (see for example, Sambrook et al., 2001, incorporated herein by reference).

In certain aspects, the present disclosure concerns a nucleic acid that is an isolated nucleic acid. As used herein, the term “isolated nucleic acid” refers to a nucleic acid molecule (e.g., an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

3. Nucleic Acid Segments

In certain embodiments, the nucleic acid is a nucleic acid segment. As used herein, the term “nucleic acid segment,” are fragments of a nucleic acid, such as, for a non-limiting example, those that encode only part of a PDE3A promoter sequence, or part of the PDE3A gene locus or gene sequence. Thus, a “nucleic acid segment” may comprise any part of a gene sequence, including from about 2 nucleotides to the full length gene including promoter regions to the polyadenylation signal and any length that includes all the coding region.

Various nucleic acid segments may be designed based on a particular nucleic acid sequence, and may be of any length. By assigning numeric values to a sequence, for example, the first residue is 1, the second residue is 2, etc., an algorithm defining all nucleic acid segments can be created:

n to n+y

where n is an integer from 1 to the last number of the sequence and y is the length of the nucleic acid segment minus one, where n+y does not exceed the last number of the sequence. Thus, for a 10-mer, the nucleic acid segments correspond to bases 1 to 10, 2 to 11, 3 to 12 . . . and so on. For a 15-mer, the nucleic acid segments correspond to bases 1 to 15, 2 to 16, 3 to 17 . . . and so on. For a 20-mer, the nucleic segments correspond to bases 1 to 20, 2 to 21, 3 to 22 . . . and so on. In certain embodiments, the nucleic acid segment may be a probe or primer. As used herein, a “probe” generally refers to a nucleic acid used in a detection method or composition. As used herein, a “primer” generally refers to a nucleic acid used in an extension or amplification method or composition.

4. Nucleic Acid Complements

The present disclosure also encompasses a nucleic acid that is complementary to a nucleic acid. A nucleic acid is “complement(s)” or is “complementary” to another nucleic acid when it is capable of base-pairing with another nucleic acid according to the standard Watson-Crick, Hoogsteen or reverse Hoogsteen binding complementarity rules. As used herein “another nucleic acid” may refer to a separate molecule or a spatial separated sequence of the same molecule. In particular embodiments, a complement is a hybridization probe or amplification primer for the detection of a nucleic acid polymorphism.

As used herein, the term “complementary” or “complement” also refers to a nucleic acid comprising a sequence of consecutive nucleobases or semiconsecutive nucleobases (e.g., one or more nucleobase moieties are not present in the molecule) capable of hybridizing to another nucleic acid strand or duplex even if less than all the nucleobases do not base pair with a counterpart nucleobase. However, in some diagnostic or detection embodiments, completely complementary nucleic acids are contemplated.

5. Nucleic Acid Detection and Evaluation

Genotyping can be performed using methods described in Small et al. (2002), which is incorporated herein by reference. It will be understood by the skilled artisan that other standard techniques are available for genotyping and any technique may be used with the present disclosure. Traditional methods of nucleic acid detection methods are provided below. Notably, in the past decade an explosion of newer DNA sequencing methodologies have been developed and are rapidly being introduced to both research and clinical arenas. Next-Generation DNA sequencing (sometimes called second-generation DNA sequencing) has already transformed the sequencing landscape and it able to do high-throughput genotyping of polymorphisms, even at a genome-wide level. These newer technologies are already anticipated to give way to third-generation DNA sequencing where individual DNA molecules are sequenced directly without any amplification or capture steps. Thus as, these technologies replace more traditional methods and genome-wide sequencing becomes the norm, it is expected that most, or possibly even all, polymorphisms will be genotyped in a single assay. As this field is in flux a detailed description of the evolving technology is not provided here.

One traditional genotyping method involves isolating from the individual a nucleic acid mixture comprising the two copies of the PDE3A gene, or a fragment thereof, that are present in the individual, and determining the nucleotide sequence or presence of a nucleotide insertion in the promoter of the PDE3A gene. Other polymorphisms, such as single nucleotide polymorphisms can be linked to and indicative of the insertion polymorphism described herein.

Those in the art will readily recognize that nucleic acid molecules may be double-stranded molecules and that reference to a particular site on one strand refers, as well, to the corresponding site on a complementary strand. Thus, in defining a polymorphic site, reference to a sequence including an adenine, a thymine (uridine), a cytosine, or a guanine at a particular site on one strand of a nucleic acid molecule is also intended to include the thymine (uridine), adenine, guanine, or cytosine (respectively) at the corresponding site on a complementary strand of a nucleic acid molecule. Thus, reference may be made to either strand and still comprise the same polymorphic site and an oligonucleotide may be designed to hybridize to either strand.

Typically, the nucleic acid mixture is isolated from a biological sample taken from the individual, such as a blood sample or tissue sample using standard techniques such as disclosed in Jones (1963) which is hereby incorporated by reference. Suitable tissue samples include whole blood, semen, saliva, tears, urine, fecal material, sweat, buccal, skin, and hair. The nucleic acid mixture may be comprised of genomic DNA.

The ability to predict a patient's response to a PDE inhibitor is useful for physicians in making decisions about how to treat a patient having heart failure. A patient whose genotype indicates the patient will probably respond well to the PDE inhibitor (i.e., a patient homozygous for a Evi-1 binding site insertional polymorphism) would be a better candidate for PDE inhibitor therapy than a patient who is likely to exhibit tolerance to PDE inhibitor therapy, or an intermediate response, or no response, and the physician would be able to determine with less trial and error which individuals should receive an alternative form of therapy.

In the genotyping methods used in the present disclosure, the identity of a polymorphic site may be determined by amplifying a target region containing the polymorphic site directly from one or both copies of the PDE3A gene present in the individual and the sequence of the amplified region(s) determined by conventional methods or evaluated directly.

The target region(s) may be amplified using any oligonucleotide-directed amplification method, including but not limited to polymerase chain reaction (PCR) (U.S. Pat. No. 4,965,188), ligase chain reaction (LCR) (Barany et al., 1991; PCT Appln. WO90/01069), and oligonucleotide ligation assay (OLA) (Landegren et al., 1988). Oligonucleotides useful as primers or probes in such methods should specifically hybridize to a region of the nucleic acid that contains or is adjacent to the polymorphic site. Typically, the oligonucleotides are between 10 and 35 nucleotides in length and preferably, between 15 and 30 nucleotides in length. Most preferably, the oligonucleotides are 20 to 25 nucleotides long. The exact length of the oligonucleotide will depend on many factors that are routinely considered and practiced by the skilled artisan.

Other known nucleic acid amplification procedures may be used to amplify the target region including transcription-based amplification systems (U.S. Pat. No. 5,130,238; EP 329,822; U.S. Pat. No. 5,169,766, PCT Appln. WO89/06700) and isothermal methods (Walker et al., 1992).

A polymorphism in the target region may also be assayed before or after amplification using one of several hybridization-based methods known in the art. Typically, allele-specific oligonucleotides are utilized in performing such methods. The allele-specific oligonucleotides may be used as differently labeled probe pairs, with one member of the pair showing a perfect match to one variant of a target sequence and the other member showing a perfect match to a different variant. In some embodiments, more than one polymorphic site may be detected at once using a set of allele-specific oligonucleotides or oligonucleotide pairs.

Hybridization of an allele-specific oligonucleotide to a target polynucleotide may be performed with both entities in solution, or such hybridization may be performed when either the oligonucleotide or the target polynucleotide is covalently or noncovalently affixed to a solid support. Attachment may be mediated, for example, by antibody-antigen interactions, poly-L-Lys, streptavidin or avidin-biotin, salt bridges, hydrophobic interactions, chemical linkages, UV cross-linking baking, etc. Allele-specific oligonucleotides may be synthesized directly on the solid support or attached to the solid support subsequent to synthesis. Solid-supports suitable for use in detection methods of the disclosure include substrates made of silicon, glass, plastic, paper and the like, which may be formed, for example, into wells (as in 96-well plates), slides, sheets, membranes, fibers, chips, dishes, and beads. The solid support may be treated, coated or derivatized to facilitate the immobilization of the allele-specific oligonucleotide or target nucleic acid.

The genotype for one or more polymorphic sites in the PDE3A gene of an individual may also be determined by hybridization of one or both copies of the gene, or a fragment thereof, to nucleic acid arrays and subarrays such as described in PCT Appln. WO 95/11995. The arrays would contain a battery of allele-specific oligonucleotides representing each of the polymorphic sites to be included in the genotype or haplotype.

The identity of polymorphisms may also be determined using a mismatch detection technique, including but not limited to the RNase protection method using riboprobes (Winter et al., 1985); Meyers et al., 1985) and proteins which recognize nucleotide mismatches, such as the E. coli mutS protein (Modrich, 1991). Alternatively, variant alleles can be identified by single strand conformation polymorphism (SSCP) analysis (Orita et al., 1989; Humphries et al., 1996) or denaturing gradient gel electrophoresis (DGGE) (Wartell et al., 1990; Sheffield et al., 1989).

A polymerase-mediated primer extension method may also be used to identify the polymorphism(s). Several such methods have been described in the patent and scientific literature. Extended primers containing a polymorphism may be detected by mass spectrometry as described in U.S. Pat. No. 5,605,798. An other primer extension method is allele-specific PCR (Ruano et al., 1989; Ruano et al., 1991; PCT Appln. WO 93/22456; Turki et al., 1995).

Polymorphic variation in the promoter of the human PDE3A gene can also be detected using differential digestion of DNA by certain restriction enzymes (Small et al., 2002) or by any other method that identifies the nucleotide insertion in the PDE3A gene.

a. Hybridization

The nucleotide sequences of the disclosure may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting a specific polymorphism. It is generally appreciated that conditions can be rendered more stringent by the addition of increasing amounts of formamide. For example, under highly stringent conditions, hybridization to filter-bound DNA may be carried out in 0.5 M NaHPO₄, 7% sodium dodecyl sulfate (SDS), 1 mM EDTA at 65° C., and washing in 0.1×SSC/0.1% SDS at 68° C. (Ausubel et al., 1989).

Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Under low stringent conditions, such as moderately stringent conditions the washing may be carried out for example in 0.2×SSC/0.1% SDS at 42° C. (Ausubel et al., 1989). Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present disclosure in combination with an appropriate means, such as a label, for determining hybridization. A wide variety of appropriate indicator means are known in the art, including fluorescent, radioactive, enzymatic or other ligands, such as avidin/biotin, which are capable of being detected. In particular embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples. In other aspects, a particular nuclease cleavage site may be present and detection of a particular nucleotide sequence can be determined by the presence or absence of nucleic acid cleavage.

In general, it is envisioned that the probes or primers described herein will be useful as reagents in solution hybridization, as in PCR, for detection of expression or genotype of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present disclosure are disclosed in U.S. Pat. Nos. 5,849,481, 5,849,486 and 5,851,772. The relevant portions of these and other references identified in this section of the Specification are incorporated herein by reference.

b. Amplification of Nucleic Acids

Nucleic acids used as a template for amplification may be isolated from cells, tissues or other samples according to standard methodologies (Sambrook et al., 2001). In certain embodiments, analysis is performed on whole cell or tissue homogenates or biological fluid samples with or without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is contemplated.

Pairs of primers designed to selectively hybridize to nucleic acids corresponding to the PDE3A gene locus, or variants thereof, and fragments thereof are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids that contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected, analyzed or quantified. In certain applications, the detection may be performed by visual means. In certain applications, the detection may involve indirect identification of the product via chemiluminescence, radioactive scintigraphy of incorporated radiolabel or fluorescent label or even via a system using electrical and/or thermal impulse signals (Affymax technology; Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

Another method for amplification is ligase chain reaction (“LCR”), disclosed in European Appln. 320 308, incorporated herein by reference in its entirety. U.S. Pat. No. 4,883,750 describes a method similar to LCR for binding probe pairs to a target sequence. A method based on PCR™ and oligonucleotide ligase assay (OLA) (described in further detail below), disclosed in U.S. Pat. No. 5,912,148, may also be used.

Alternative methods for amplification of target nucleic acid sequences that may be used in the practice of the present disclosure are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, Great Britain Appln. 2 202 328, and in PCT Application PCT/US89/01025, each of which is incorporated herein by reference in its entirety. Qbeta Replicase, described in PCT Application PCT/US87/00880, may also be used as an amplification method in the present disclosure.

An isothermal amplification method, in which restriction endonucleases and ligases are used to achieve the amplification of target molecules that contain nucleotide 5′-[alpha-thio]-triphosphates in one strand of a restriction site, may also be useful in the amplification of nucleic acids in the present disclosure (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; PCT Appln. WO 88/10315, incorporated herein by reference in their entirety). European Patent Appln. 329 822 disclose a nucleic acid amplification process involving cyclically synthesizing single-stranded RNA (“ssRNA”), ssDNA, and double-stranded DNA (dsDNA), which may be used in accordance with the present disclosure.

PCT Appln. WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989).

c. Detection of Nucleic Acids

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 2001). Separated amplification products may be cut out and eluted from the gel for further manipulation. Using low melting point agarose gels, the separated band may be removed by heating the gel, followed by extraction of the nucleic acid.

Separation of nucleic acids may also be effected by spin columns and/or chromatographic techniques known in art. There are many kinds of chromatography which may be used in the practice of the present disclosure, including adsorption, partition, ion-exchange, hydroxylapatite, molecular sieve, reverse-phase, column, paper, thin-layer, and gas chromatography as well as HPLC.

In certain embodiments, the amplification products are visualized, with or without separation. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 2001). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present disclosure.

Other methods of nucleic acid detection that may be used in the practice of the instant disclosure are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

d. Other Assays

Other methods for genetic screening may be used within the scope of the present disclosure, for example, to detect mutations in genomic DNA, cDNA and/or RNA samples. Methods used to detect point mutations include denaturing gradient gel electrophoresis (“DGGE”), restriction fragment length polymorphism analysis (“RFLP”), chemical or enzymatic cleavage methods, direct sequencing of target regions amplified by PCR™ (see above), single-strand conformation polymorphism analysis (“SSCP”) and other methods well known in the art.

One method of screening for point mutations is based on RNase cleavage of base pair mismatches in RNA/DNA or RNA/RNA heteroduplexes. As used herein, the term “mismatch” is defined as a region of one or more unpaired or mispaired nucleotides in a double-stranded RNA/RNA, RNA/DNA or DNA/DNA molecule. This definition thus includes mismatches due to insertion/deletion mutations, as well as single or multiple base point mutations.

U.S. Pat. No. 4,946,773 describes an RNase A mismatch cleavage assay that involves annealing single-stranded DNA or RNA test samples to an RNA probe, and subsequent treatment of the nucleic acid duplexes with RNase A. For the detection of mismatches, the single-stranded products of the RNase A treatment, electrophoretically separated according to size, are compared to similarly treated control duplexes. Samples containing smaller fragments (cleavage products) not seen in the control duplex are scored as positive.

Other investigators have described the use of RNase I in mismatch assays. The use of RNase I for mismatch detection is described in literature from Promega Biotech. Promega markets a kit containing RNase I that is reported to cleave three out of four known mismatches. Others have described using the MutS protein or other DNA-repair enzymes for detection of single-base mismatches.

Alternative methods for detection of deletion, insertion or substitution mutations that may be used in the practice of the present disclosure are disclosed in U.S. Pat. Nos. 5,849,483, 5,851,770, 5,866,337, 5,925,525 and 5,928,870, each of which is incorporated herein by reference in its entirety.

e. Polymorphism Nucleic Acid Screening Methods

Spontaneous mutations that arise during the course of evolution in the genomes of organisms are often not immediately transmitted throughout all of the members of the species, thereby creating polymorphic alleles that co-exist in the species populations. Often polymorphisms are the cause of genetic diseases. Several classes of polymorphisms have been identified. For example, variable nucleotide type polymorphisms (VNTRs), arise from spontaneous tandem duplications of di- or trinucleotide repeated motifs of nucleotides. If such variations alter the lengths of DNA fragments generated by restriction endonuclease cleavage, the variations are referred to as restriction fragment length polymorphisms (RFLPs). RFLPs are been widely used in human and animal genetic analyses.

Another class of polymorphisms are generated by the replacement of a single nucleotide. Such single nucleotide polymorphisms (SNPs) rarely result in changes in a restriction endonuclease site. Thus, SNPs are rarely detectable restriction fragment length analysis. SNPs are the most common genetic variations and occur once every 100 to 300 bases and several SNP mutations have been found that affect a single nucleotide in a protein-encoding gene in a manner sufficient to actually cause a genetic disease. SNP diseases are exemplified by hemophilia, sickle-cell anemia, hereditary hemochromatosis, late-onset Alzheimer's disease, etc.

Several methods have been developed to screen polymorphisms and some examples are listed below. The reference of Kwok and Chen (2003) and Kwok (2001) provide overviews of some of these methods; both of these references are specifically incorporated by reference.

SNPs relating to ABCC2 can be characterized by the use of any of these methods or suitable modification thereof. Such methods include the direct or indirect sequencing of the site, the use of restriction enzymes where the respective alleles of the site create or destroy a restriction site, the use of allele-specific hybridization probes, the use of antibodies that are specific for the proteins encoded by the different alleles of the polymorphism, or any other biochemical interpretation.

DNA Sequencing. The most commonly used method of characterizing a polymorphism is direct DNA sequencing of the genetic locus that flanks and includes the polymorphism. Such analysis can be accomplished using either the “dideoxy-mediated chain termination method,” also known as the “Sanger Method” (Sanger et al., 1975) or the “chemical degradation method,” also known as the “Maxam-Gilbert method” (Maxam et al., 1977). Sequencing in combination with genomic sequence-specific amplification technologies, such as the polymerase chain reaction may be utilized to facilitate the recovery of the desired genes (Mullis et al., 1986; European Patent Appln. 50,424; European Patent Appln. 84,796, European Patent Appln. 258,017, European Patent Appln. 237,362; European Patent Appln. 201,184; U.S. Pat. Nos. 4,683,202; 4,582,788; and 4,683,194), all of the above incorporated herein by reference.

Exonuclease Resistance.

Other methods that can be employed to determine the identity of a nucleotide present at a polymorphic site utilize a specialized exonuclease-resistant nucleotide derivative (U.S. Pat. No. 4,656,127). A primer complementary to an allelic sequence immediately 3′- to the polymorphic site is hybridized to the DNA under investigation. If the polymorphic site on the DNA contains a nucleotide that is complementary to the particular exonucleotide-resistant nucleotide derivative present, then that derivative will be incorporated by a polymerase onto the end of the hybridized primer. Such incorporation makes the primer resistant to exonuclease cleavage and thereby permits its detection. As the identity of the exonucleotide-resistant derivative is known one can determine the specific nucleotide present in the polymorphic site of the DNA.

Microsequencing Methods.

Several other primer-guided nucleotide incorporation procedures for assaying polymorphic sites in DNA have been described (Komher et al., 1989; Sokolov, 1990; Syvanen 1990; Kuppuswamy et al., 1991; Prezant et al., 1992; Ugozzoll et al., 1992; Nyren et al., 1993). These methods rely on the incorporation of labeled deoxynucleotides to discriminate between bases at a polymorphic site. As the signal is proportional to the number of deoxynucleotides incorporated, polymorphisms that occur in runs of the same nucleotide result in a signal that is proportional to the length of the run (Syvanen et al., 1990).

Extension in Solution.

French Patent 2,650,840 and PCT Appln. WO91/02087 discuss a solution-based method for determining the identity of the nucleotide of a polymorphic site. According to these methods, a primer complementary to allelic sequences immediately 3′- to a polymorphic site is used. The identity of the nucleotide of that site is determined using labeled dideoxynucleotide derivatives which are incorporated at the end of the primer if complementary to the nucleotide of the polymorphic site.

Genetic Bit Analysis or Solid-Phase Extension.

PCT Appln. WO92/15712 describes a method that uses mixtures of labeled terminators and a primer that is complementary to the sequence 3′ to a polymorphic site. The labeled terminator that is incorporated is complementary to the nucleotide present in the polymorphic site of the target molecule being evaluated and is thus identified. Here the primer or the target molecule is immobilized to a solid phase.

Oligonucleotide Ligation Assay (OLA).

This is another solid phase method that uses different methodology (Landegren et al., 1988). Two oligonucleotides, capable of hybridizing to abutting sequences of a single strand of a target DNA are used. One of these oligonucleotides is biotinylated while the other is detectably labeled. If the precise complementary sequence is found in a target molecule, the oligonucleotides will hybridize such that their termini abut, and create a ligation substrate. Ligation permits the recovery of the labeled oligonucleotide by using avidin. Other nucleic acid detection assays, based on this method, combined with PCR have also been described (Nickerson et al., 1990). Here, PCR is used to achieve the exponential amplification of target DNA, which is then detected using the OLA.

Ligase/Polymerase-Mediated Genetic Bit Analysis.

U.S. Pat. No. 5,952,174 describes a method that also involves two primers capable of hybridizing to abutting sequences of a target molecule. The hybridized product is formed on a solid support to which the target is immobilized. Here the hybridization occurs such that the primers are separated from one another by a space of a single nucleotide. Incubating this hybridized product in the presence of a polymerase, a ligase, and a nucleoside triphosphate mixture containing at least one deoxynucleoside triphosphate allows the ligation of any pair of abutting hybridized oligonucleotides. Addition of a ligase results in two events required to generate a signal, extension and ligation. This provides a higher specificity and lower “noise” than methods using either extension or ligation alone and unlike the polymerase-based assays, this method enhances the specificity of the polymerase step by combining it with a second hybridization and a ligation step for a signal to be attached to the solid phase.

Invasive Cleavage Reactions.

Invasive cleavage reactions can be used to evaluate cellular DNA for a particular polymorphism. A technology called INVADER® employs such reactions (e.g., de Arruda et al., 2002; Stevens et al., 2003, which are incorporated by reference). Generally, there are three nucleic acid molecules: 1) an oligonucleotide upstream of the target site (“upstream oligo”), 2) a probe oligonucleotide covering the target site (“probe”), and 3) a single-stranded DNA with the target site (“target”). The upstream oligo and probe do not overlap but they contain contiguous sequences. The probe contains a donor fluorophore, such as fluoroscein, and an acceptor dye, such as Dabcyl. The nucleotide at the 3′ terminal end of the upstream oligo overlaps (“invades”) the first base pair of a probe-target duplex. Then the probe is cleaved by a structure-specific 5′ nuclease causing separation of the fluorophore/quencher pair, which increases the amount of fluorescence that can be detected. See Lu et al., 2004. In some cases, the assay is conducted on a solid-surface or in an array format.

Other Methods to Detect SNPs.

Several other specific methods for polymorphism detection and identification are presented below and may be used as such or with suitable modifications in conjunction with identifying polymorphisms of the PDE3A gene in the present disclosure. Several other methods are also described on the SNP web site of the NCBI on the World Wide Web at ncbi.nlm.nih.gov/SNP, incorporated herein by reference.

In a particular embodiment, extended haplotypes may be determined at any given locus in a population, which allows one to identify exactly which SNPs will be redundant and which will be essential in association studies. The latter is referred to as ‘haplotype tag SNPs (htSNPs)’, markers that capture the haplotypes of a gene or a region of linkage disequilibrium. See Johnson et al. (2001) and Ke and Cardon (2003), each of which is incorporated herein by reference, for exemplary methods.

The VDA-assay utilizes PCR amplification of genomic segments by long PCR methods using TaKaRa LA Taq reagents and other standard reaction conditions. The long amplification can amplify DNA sizes of about 2,000-12,000 bp. Hybridization of products to variant detector array (VDA) can be performed by a Affymetrix High Throughput Screening Center and analyzed with computerized software.

A method called Chip Assay uses PCR amplification of genomic segments by standard or long PCR protocols. Hybridization products are analyzed by VDA, Halushka et al. (1999), incorporated herein by reference. SNPs are generally classified as “Certain” or “Likely” based on computer analysis of hybridization patterns. By comparison to alternative detection methods such as nucleotide sequencing, “Certain” SNPs have been confirmed 100% of the time; and “likely” SNPs have been confirmed 73% of the time by this method.

Other methods simply involve PCR amplification following digestion with the relevant restriction enzyme. Yet others involve sequencing of purified PCR products from known genomic regions.

In yet another method, individual exons or overlapping fragments of large exons are PCR-amplified. Primers are designed from published or database sequences and PCR-amplification of genomic DNA is performed using the following conditions: 200 ng DNA template, 0.5 μM each primer, 80 μM each of dCTP, dATP, dTTP and dGTP, 5% formamide, 1.5 mM MgCl₂, 0.5 U of Taq polymerase and 0.1 volume of the Taq buffer. Thermal cycling is performed and resulting PCR-products are analyzed by PCR-single strand conformation polymorphism (PCR-SSCP) analysis, under a variety of conditions, e.g., 5 or 10% polyacrylamide gel with 15% urea, with or without 5% glycerol. Electrophoresis is performed overnight. PCR-products that show mobility shifts are reamplified and sequenced to identify nucleotide variation.

In a method called CGAP-GAI (DEMIGLACE), sequence and alignment data (from a PHRAP.ace file), quality scores for the sequence base calls (from PHRED quality files), distance information (from PHYLIP dnadist and neighbour programs) and base-calling data (from PHRED ‘-d’ switch) are loaded into memory. Sequences are aligned and examined for each vertical chunk (‘slice’) of the resulting assembly for disagreement. Any such slice is considered a candidate SNP (DEMIGLACE). A number of filters are used by DEMIGLACE to eliminate slices that are not likely to represent true polymorphisms. These include filters that: (i) exclude sequences in any given slice from SNP consideration where neighboring sequence quality scores drop 40% or more; (ii) exclude calls in which peak amplitude is below the fifteenth percentile of all base calls for that nucleotide type; (iii) disqualify regions of a sequence having a high number of disagreements with the consensus from participating in SNP calculations; (iv) removed from consideration any base call with an alternative call in which the peak takes up 25% or more of the area of the called peak; (v) exclude variations that occur in only one read direction. PHRED quality scores were converted into probability-of-error values for each nucleotide in the slice. Standard Baysian methods are used to calculate the posterior probability that there is evidence of nucleotide heterogeneity at a given location.

In a method called CU-RDF (RESEQ), PCR amplification is performed from DNA isolated from blood using specific primers for each SNP, and after typical cleanup protocols to remove unused primers and free nucleotides, direct sequencing using the same or nested primers.

In a method called DEBNICK (METHOD-B), a comparative analysis of clustered EST sequences is performed and confirmed by fluorescent-based DNA sequencing. In a related method, called DEBNICK (METHOD-C), comparative analysis of clustered EST sequences with phred quality >20 at the site of the mismatch, average phred quality ≧20 over 5 bases 5′-FLANK and 3′ to the SNP, no mismatches in 5 bases 5′ and 3′ to the SNP, at least two occurrences of each allele is performed and confirmed by examining traces.

In a method identified by ERO (RESEQ), new primers sets are designed for electronically published STSs and used to amplify DNA from 10 different mouse strains. The amplification product from each strain is then gel purified and sequenced using a standard dideoxy, cycle sequencing technique with ³²P-labeled terminators. All the ddATP terminated reactions are then loaded in adjacent lanes of a sequencing gel followed by all of the ddGTP reactions and so on. SNPs are identified by visually scanning the radiographs.

In another method identified as ERO (RESEQ-HT), new primers sets are designed for electronically published murine DNA sequences and used to amplify DNA from 10 different mouse strains. The amplification product from each strain is prepared for sequencing by treating with Exonuclease I and Shrimp Alkaline Phosphatase. Sequencing is performed using ABI Prism Big Dye Terminator Ready Reaction Kit (Perkin-Elmer) and sequence samples are run on the 3700 DNA Analyzer (96 Capillary Sequencer).

FGU-CBT (SCA2-SNP) identifies a method where the region containing the SNP is PCR amplified using the primers SCA2-FP3 and SCA2-RP3. Approximately 100 ng of genomic DNA is amplified in a 50 ml reaction volume containing a final concentration of 5 mM Tris, 25 mM KCl, 0.75 mM MgCl₂, 0.05% gelatin, 20 pmol of each primer and 0.5 U of Taq DNA polymerase. Samples are denatured, annealed and extended and the PCR product is purified from a band cut out of the agarose gel using, for example, the QlAquick gel extraction kit (Qiagen) and is sequenced using dye terminator chemistry on an ABI Prism 377 automated DNA sequencer with the PCR primers.

In a method identified as JBLACK (SEQ/RESTRICT), two independent PCR reactions are performed with genomic DNA. Products from the first reaction are analyzed by sequencing, indicating a unique FspI restriction site. The mutation is confirmed in the product of the second PCR reaction by digesting with Fsp I.

In a method described as KWOK(1), SNPs are identified by comparing high quality genomic sequence data from four randomly chosen individuals by direct DNA sequencing of PCR products with dye-terminator chemistry (see Kwok et al., 1996). In a related method identified as KWOK(2) SNPs are identified by comparing high quality genomic sequence data from overlapping large-insert clones such as bacterial artificial chromosomes (BACs) or P1-based artificial chromosomes (PACs). An STS containing this SNP is then developed and the existence of the SNP in various populations is confirmed by pooled DNA sequencing (see Taillon-Miller et al., 1998). In another similar method called KWOK(3), SNPs are identified by comparing high quality genomic sequence data from overlapping large-insert clones BACs or PACs. The SNPs found by this approach represent DNA sequence variations between the two donor chromosomes but the allele frequencies in the general population have not yet been determined. In method KWOK(5), SNPs are identified by comparing high quality genomic sequence data from a homozygous DNA sample and one or more pooled DNA samples by direct DNA sequencing of PCR products with dye-terminator chemistry. The STSs used are developed from sequence data found in publicly available databases. Specifically, these STSs are amplified by PCR against a complete hydatidiform mole (CHM) that has been shown to be homozygous at all loci and a pool of DNA samples from 80 CEPH parents (see Kwok et al., 1994).

In another such method, KWOK (OverlapSnpDetectionWithPolyBayes), SNPs are discovered by automated computer analysis of overlapping regions of large-insert human genomic clone sequences. For data acquisition, clone sequences are obtained directly from large-scale sequencing centers. This is necessary because base quality sequences are not present/available through GenBank. Raw data processing involves analyzed of clone sequences and accompanying base quality information for consistency. Finished (‘base perfect’, error rate lower than 1 in 10,000 bp) sequences with no associated base quality sequences are assigned a uniform base quality value of 40 (1 in 10,000 bp error rate). Draft sequences without base quality values are rejected. Processed sequences are entered into a local database. A version of each sequence with known human repeats masked is also stored. Repeat masking is performed with the program “MASKERAID.” Overlap detection: Putative overlaps are detected with the program “WUBLAST.” Several filtering steps followed in order to eliminate false overlap detection results, i.e. similarities between a pair of clone sequences that arise due to sequence duplication as opposed to true overlap. Total length of overlap, overall percent similarity, and number of sequence differences between nucleotides with high base quality value “high-quality mismatches are considered. Results are also compared to results of restriction fragment mapping of genomic clones at Washington University Genome Sequencing Center, finisher's reports on overlaps, and results of the sequence contig building effort at the NCBI. SNP detection: Overlapping pairs of clone sequence are analyzed for candidate SNP sites with the ‘POLYBAYES’ SNP detection software. Sequence differences between the pair of sequences are scored for the probability of representing true sequence variation as opposed to sequencing error. This process requires the presence of base quality values for both sequences. High-scoring candidates are extracted. The search is restricted to substitution-type single base pair variations. Confidence score of candidate SNP is computed by the POLYBAYES software.

In method identified by KWOK (TaqMan assay), the TaqMan assay is used to determine genotypes for 90 random individuals. In method identified by KYUGEN(Q1), DNA samples of indicated populations are pooled and analyzed by PLACE-SSCP. Peak heights of each allele in the pooled analysis are corrected by those in a heterozygote, and are subsequently used for calculation of allele frequencies. Allele frequencies higher than 10% are reliably quantified by this method. Allele frequency=0 (zero) means that the allele was found among individuals, but the corresponding peak is not seen in the examination of pool. Allele frequency=0-0.1 indicates that minor alleles are detected in the pool but the peaks are too low to reliably quantify.

In yet another method identified as KYUGEN (Method1), PCR products are post-labeled with fluorescent dyes and analyzed by an automated capillary electrophoresis system under SSCP conditions (PLACE-SSCP). Four or more individual DNAs are analyzed with or without two pooled DNA (Japanese pool and CEPH parents' pool) in a series of experiments. Alleles are identified by visual inspection. Individual DNAs with different genotypes are sequenced and SNPs identified. Allele frequencies are estimated from peak heights in the pooled samples after correction of signal bias using peak heights in heterozygotes. For the PCR primers are tagged to have 5′-ATT or 5′-GTT at their ends for post-labeling of both strands. Samples of DNA (10 ng/μl) are amplified in reaction mixtures containing the buffer (10 mM Tris-HCl, pH 8.3 or 9.3, 50 mM KCl, 2.0 mM MgCl₂), 0.25 μM of each primer, 200 μM of each dNTP, and 0.025 units/μl of Taq DNA polymerase premixed with anti-Taq antibody. The two strands of PCR products are differentially labeled with nucleotides modified with R110 and R6G by an exchange reaction of Klenow fragment of DNA polymerase I. The reaction is stopped by adding EDTA, and unincorporated nucleotides are dephosphorylated by adding calf intestinal alkaline phosphatase. For the SSCP: an aliquot of fluorescently labeled PCR products and TAMRA-labeled internal markers are added to deionized formamide, and denatured. Electrophoresis is performed in a capillary using an ABI Prism 310 Genetic Analyzer. Genescan softwares (P-E Biosystems) are used for data collection and data processing. DNA of individuals (two to eleven) including those who showed different genotypes on SSCP are subjected for direct sequencing using big-dye terminator chemistry, on ABI Prism 310 sequencers. Multiple sequence trace files obtained from ABI Prism 310 are processed and aligned by Phred/Phrap and viewed using Consed viewer. SNPs are identified by PolyPhred software and visual inspection.

In yet another method identified as KYUGEN (Method2), individuals with different genotypes are searched by denaturing HPLC (DHPLC) or PLACE-SSCP (Inazuka et al., 1997) and their sequences are determined to identify SNPs. PCR is performed with primers tagged with 5′-ATT or 5′-GTT at their ends for post-labeling of both strands. DHPLC analysis is carried out using the WAVE DNA fragment analysis system (Transgenomic). PCR products are injected into DNASep column, and separated under the conditions determined using WAVEMaker program (Transgenomic). The two strands of PCR products that are differentially labeled with nucleotides modified with R110 and R6G by an exchange reaction of Klenow fragment of DNA polymerase I. The reaction is stopped by adding EDTA, and unincorporated nucleotides are dephosphorylated by adding calf intestinal alkaline phosphatase. SSCP followed by electrophoresis is performed in a capillary using an ABI Prism 310 Genetic Analyzer. Genescan softwares (P-E Biosystems). DNA of individuals including those who showed different genotypes on DHPLC or SSCP are subjected for direct sequencing using big-dye terminator chemistry, on ABI Prism 310 sequencer. Multiple sequence trace files obtained from ABI Prism 310 are processed and aligned by Phred/Phrap and viewed using Consed viewer. SNPs are identified by PolyPhred software and visual inspection. Trace chromatogram data of EST sequences in Unigene are processed with PHRED. To identify likely SNPs, single base mismatches are reported from multiple sequence alignments produced by the programs PHRAP, BRO and POA for each Unigene cluster. BRO corrected possible misreported EST orientations, while POA identified and analyzed non-linear alignment structures indicative of gene mixing/chimeras that might produce spurious SNPs. Bayesian inference is used to weigh evidence for true polymorphism versus sequencing error, misalignment or ambiguity, misclustering or chimeric EST sequences, assessing data such as raw chromatogram height, sharpness, overlap and spacing; sequencing error rates; context-sensitivity; cDNA library origin, etc.

In method identified as MARSHFIELD (Method-B), overlapping human DNA sequences which contained putative insertion/deletion polymorphisms are identified through searches of public databases. PCR primers which flanked each polymorphic site are selected from the consensus sequences. Primers are used to amplify individual or pooled human genomic DNA. Resulting PCR products are resolved on a denaturing polyacrylamide gel and a PhosphorImager is used to estimate allele frequencies from DNA pools.

f. Next-Gen Sequencing Methods

Massively Parallel Signature Sequencing (MPSS).

The first of the next-generation sequencing technologies, massively parallel signature sequencing (or MPSS), was developed in the 1990s at Lynx Therapeutics. MPSS was a bead-based method that used a complex approach of adapter ligation followed by adapter decoding, reading the sequence in increments of four nucleotides. This method made it susceptible to sequence-specific bias or loss of specific sequences. Because the technology was so complex, MPSS was only performed ‘in-house’ by Lynx Therapeutics and no DNA sequencing machines were sold to independent laboratories. Lynx Therapeutics merged with Solexa (later acquired by Illumina) in 2004, leading to the development of sequencing-by-synthesis, a simpler approach acquired from Manteia Predictive Medicine, which rendered MPSS obsolete. However, the essential properties of the MPSS output were typical of later “next-generation” data types, including hundreds of thousands of short DNA sequences. In the case of MPSS, these were typically used for sequencing cDNA for measurements of gene expression levels. Indeed, the powerful Illumina HiSeq2000, HiSeq2500 and MiSeq systems are based on MPSS.

Polony sequencing.

The Polony sequencing method, developed in the laboratory of George M. Church at Harvard, was among the first next-generation sequencing systems and was used to sequence a full genome in 2005. It combined an in vitro paired-tag library with emulsion PCR, an automated microscope, and ligation-based sequencing chemistry to sequence an E. coli genome at an accuracy of >99.9999% and a cost approximately 1/9 that of Sanger sequencing. The technology was licensed to Agencourt Biosciences, subsequently spun out into Agencourt Personal Genomics, and eventually incorporated into the Applied Biosystems SOLiD platform, which is now owned by Life Technologies.

454 Pyrosequencing.

A parallelized version of pyrosequencing was developed by 454 Life Sciences, which has since been acquired by Roche Diagnostics. The method amplifies DNA inside water droplets in an oil solution (emulsion PCR), with each droplet containing a single DNA template attached to a single primer-coated bead that then forms a clonal colony. The sequencing machine contains many picoliter-volume wells each containing a single bead and sequencing enzymes. Pyrosequencing uses luciferase to generate light for detection of the individual nucleotides added to the nascent DNA, and the combined data are used to generate sequence read-outs. This technology provides intermediate read length and price per base compared to Sanger sequencing on one end and Solexa and SOLiD on the other.

Illumina (Solexa) Sequencing.

Solexa, now part of Illumina, developed a sequencing method based on reversible dye-terminators technology, and engineered polymerases, that it developed internally. The terminated chemistry was developed internally at Solexa and the concept of the Solexa system was invented by Balasubramanian and Klennerman from Cambridge University's chemistry department. In 2004, Solexa acquired the company Manteia Predictive Medicine in order to gain a massivelly parallel sequencing technology based on “DNA Clusters”, which involves the clonal amplification of DNA on a surface. The cluster technology was co-acquired with Lynx Therapeutics of California. Solexa Ltd. later merged with Lynx to form Solexa Inc.

In this method, DNA molecules and primers are first attached on a slide and amplified with polymerase so that local clonal DNA colonies, later coined “DNA clusters”, are formed. To determine the sequence, four types of reversible terminator bases (RT-bases) are added and non-incorporated nucleotides are washed away. A camera takes images of the fluorescently labeled nucleotides, then the dye, along with the terminal 3′ blocker, is chemically removed from the DNA, allowing for the next cycle to begin. Unlike pyrosequencing, the DNA chains are extended one nucleotide at a time and image acquisition can be performed at a delayed moment, allowing for very large arrays of DNA colonies to be captured by sequential images taken from a single camera.

Decoupling the enzymatic reaction and the image capture allows for optimal throughput and theoretically unlimited sequencing capacity. With an optimal configuration, the ultimately reachable instrument throughput is thus dictated solely by the analog-to-digital conversion rate of the camera, multiplied by the number of cameras and divided by the number of pixels per DNA colony required for visualizing them optimally (approximately 10 pixels/colony). In 2012, with cameras operating at more than 10 MHz A/D conversion rates and available optics, fluidics and enzymatics, throughput can be multiples of 1 million nucleotides/second, corresponding roughly to one human genome equivalent at 1× coverage per hour per instrument, and one human genome re-sequenced (at approx. 30×) per day per instrument (equipped with a single camera).

SOLiD Sequencing.

Applied Biosystems' (now a Life Technologies brand) SOLiD technology employs sequencing by ligation. Here, a pool of all possible oligonucleotides of a fixed length are labeled according to the sequenced position. Oligonucleotides are annealed and ligated; the preferential ligation by DNA ligase for matching sequences results in a signal informative of the nucleotide at that position. Before sequencing, the DNA is amplified by emulsion PCR. The resulting beads, each containing single copies of the same DNA molecule, are deposited on a glass slide. The result is sequences of quantities and lengths comparable to Illumina sequencing. This sequencing by ligation method has been reported to have some issue sequencing palindromic sequences.

Ion Torrent Semiconductor Sequencing.

Ion Torrent Systems Inc. (now owned by Life Technologies) developed a system based on using standard sequencing chemistry, but with a novel, semiconductor based detection system. This method of sequencing is based on the detection of hydrogen ions that are released during the polymerization of DNA, as opposed to the optical methods used in other sequencing systems. A microwell containing a template DNA strand to be sequenced is flooded with a single type of nucleotide. If the introduced nucleotide is complementary to the leading template nucleotide it is incorporated into the growing complementary strand. This causes the release of a hydrogen ion that triggers a hypersensitive ion sensor, which indicates that a reaction has occurred. If homopolymer repeats are present in the template sequence multiple nucleotides will be incorporated in a single cycle. This leads to a corresponding number of released hydrogens and a proportionally higher electronic signal.

DNA Nanoball Sequencing.

DNA nanoball sequencing is a type of high throughput sequencing technology used to determine the entire genomic sequence of an organism. The company Complete Genomics uses this technology to sequence samples submitted by independent researchers. The method uses rolling circle replication to amplify small fragments of genomic DNA into DNA nanoballs. Unchained sequencing by ligation is then used to determine the nucleotide sequence. This method of DNA sequencing allows large numbers of DNA nanoballs to be sequenced per run and at low reagent costs compared to other next generation sequencing platforms. However, only short sequences of DNA are determined from each DNA nanoball which makes mapping the short reads to a reference genome difficult. This technology has been used for multiple genome sequencing projects and is scheduled to be used for more.

Heliscope Single Molecule Sequencing.

Heliscope sequencing is a method of single-molecule sequencing developed by Helicos Biosciences. It uses DNA fragments with added poly-A tail adapters which are attached to the flow cell surface. The next steps involve extension-based sequencing with cyclic washes of the flow cell with fluorescently labeled nucleotides (one nucleotide type at a time, as with the Sanger method). The reads are performed by the Heliscope sequencer. The reads are short, up to 55 bases per run, but recent improvements allow for more accurate reads of stretches of one type of nucleotides. This sequencing method and equipment were used to sequence the genome of the M13 bacteriophage.

Single Molecule Real Time (SMRT) Sequencing.

SMRT sequencing is based on the sequencing by synthesis approach. The DNA is synthesized in zero-mode wave-guides (ZMWs)—small well-like containers with the capturing tools located at the bottom of the well. The sequencing is performed with use of unmodified polymerase (attached to the ZMW bottom) and fluorescently labelled nucleotides flowing freely in the solution. The wells are constructed in a way that only the fluorescence occurring by the bottom of the well is detected. The fluorescent label is detached from the nucleotide at its incorporation into the DNA strand, leaving an unmodified DNA strand. According to Pacific Biosciences, the SMRT technology developer, this methodology allows detection of nucleotide modifications (such as cytosine methylation). This happens through the observation of polymerase kinetics. This approach allows reads of 20,000 nucleotides or more, with average read lengths of 5 kilobases.

g. Linkage Disequilibrium

Polymorphisms in linkage disequilibrium with another polymorphism permit identification of one polymorphism that is predictive of the identity of the linked polymorphism. “Linkage disequilibrium” (“LD” as used herein, though also referred to as “LED” in the art) refers to a situation where a particular combination of alleles (i.e., a variant form of a given gene) or polymorphisms at two loci appears more frequently than would be expected by chance. “Significant” as used in respect to linkage disequilibrium, as determined by one of skill in the art, is contemplated to be a statistical p or a value that may be 0.25 or 0.1 and may be 0.1, 0.05. 0.001, 0.00001 or less. Insertions/deletions in the PDE3A promoter may be determined by evaluating the nucleic acid sequence of a polymorphism in linkage disequilibrium with the insertion/deletion. The disclosure may be implemented in this manner with respect to one or more polymorphisms so as to allow haplotype analysis. “Haplotype” is used according to its plain and ordinary meaning to one skilled in the art. It refers to a collective genotype of two or more alleles or polymorphisms along one of the homologous chromosomes.

Pharmacogenomics allows a clinician or physician to target prophylactic or therapeutic treatments to individuals who will most benefit from the treatment and to avoid treatment of individuals who will experience symptomatic side effects. A determination of PDE3A polymorphism is predictive of an individual's likelihood of developing heart failure can be accomplished by determining the genotype of the individual in the PDE3A gene, as described above. Information generated from one or more of these approaches can be used to determine appropriate dosage and treatment regimens for prophylactic or therapeutic treatment of an individual.

### III. METHODS OF TREATING CARDIAC HYPERTROPHY

Once the PDE3A promoter genotype of the individual is determined, a physician can prepare a treatment plan for the individual. Under certain circumstances maneuvers that increase myocardial cAMP prevent or reduce pathologic hypertrophy (Takahashi et al., Circulation. 2006; 114:388-396.; Sucharov et al., J Card Fail. 2011; 17:592-600.) In the event of DEL genotype (homozygous or heterozygous), the individual may be treated prophylactically to prevent or delay onset of heart failure using an agent other than a PDE3A inhibitor. As explained below, the DEL genotype indicates that an individual is not likely to demonstrate continued clinical response to a PDE3A inhibitor. Instead, a better option to achieve a clinical response for such individuals is a different therapy, such as a beta blocker, which may also increase myocardial cAMP levels (Iaccarino et al., Circulation 1998 Oct. 27; 98(17):1783-9.) In this context, the term “clinical response” means a quantitative measure of the efficacy or potency of the therapy and adverse events (i.e., side effects).

Alternatively, individuals homozygous for an insertional polymorphism in the PDE3A gene having or suspected of having or at risk of developing heart failure can be placed on a therapy that includes PDE3A inhibitors such as but not limited to enoximone. The PDE3A inhibitor may be administered alone or in combination with at least one other agent, such as a stabilizing compound.

A. Routes of Administration

Administration of agents may be by any number of routes including, but not limited to oral, intravenous, intramuscular, intra-arterial, intramedullary, intrathecal, intraventricular, intradermal, intratracheal, intravesicle, intraocular, transdermal, subcutaneous, intraperitoneal, intranasal, enteral, topical, sublingual, or rectal. Further details on techniques for formulation and administration may be found in the latest edition of Remington's Pharmaceutical Sciences (Maack Publishing Co., Easton, Pa.). In certain embodiments, the agents are formulated for oral administration.

B. Formulations

Where clinical applications are contemplated, pharmaceutical compositions will be prepared in a form appropriate for the intended application. Generally, this will entail preparing compositions that are essentially free of pyrogens, as well as other impurities that could be harmful to humans or animals.

Aqueous compositions of the present disclosure comprise an effective amount of the agent, dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present disclosure, its use in therapeutic compositions is contemplated. Supplementary active ingredients also can be incorporated into the compositions, provided they do not inactivate the vectors or cells of the compositions.

The active compositions of the present disclosure may include classic pharmaceutical preparations. Administration of these compositions according to the present disclosure may be via any common route so long as the target tissue is available via that route. This includes oral, nasal, or buccal. Alternatively, administration may be by intradermal, subcutaneous, intramuscular, intraperitoneal or intravenous injection, or by direct injection into cardiac tissue. Such compositions would normally be administered as pharmaceutically acceptable compositions, as described supra.

The active compounds may also be administered parenterally or intraperitoneally. By way of illustration, solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations generally contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

The compositions of the present disclosure generally may be formulated in a neutral or salt form. Pharmaceutically-acceptable salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like. Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like.

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Another aspect of this disclosure provides methods of treating heart failure patients by delivering agents to a patient as a controlled release formulation. As used herein, the terms “controlled,” “extended,” “sustained,” or “prolonged” release of the composition of the present disclosure will collectively be referred to herein as “controlled release,” and includes continuous or discontinuous, and linear or non-linear release of the composition of the present disclosure.

A controlled release tablet suitable for purposes of this disclosure is disclosed in U.S. Pat. No. 5,126,145, which is incorporated by reference herein. This tablet comprises, in admixture, about 5-30% high viscosity hydroxypropyl methyl cellulose, about 2-15% of a water-soluble pharmaceutical binder, about 2-20% of a hydrophobic component such as a waxy material, e.g., a fatty acid, and about 30-90% active ingredient.

This disclosure further provides a prophylaxis for or method of treating a patient having a DEL polymorphism in the PDE3A promoter following an invasive cardiac procedure comprising administering biodegradable, biocompatible polymeric film comprising agent to a patient. The polymeric films are thin compared to their length and breadth. The films typically have a uniform selected thickness between about 60 micrometers and about 5 mm. Films of between about 600 micrometers and 1 mm and between about 1 mm and about 5 mm thick, as well as films between about 60 micrometers and about 1000 micrometers, and between about 60 and about 300 micrometers are useful in the manufacture of therapeutic implants for insertion into a patient's body. The films can be administered to the patient in a manner similar to methods used in adhesion surgeries.

Either biodegradable or nonbiodegradable polymers may be used to fabricate implants in which the agent is uniformly distributed throughout the polymer matrix. A number of suitable biodegradable polymers for use in making the biodegradable films of this disclosure are known to the art, including polyanhydrides and aliphatic polyesters, preferably polylactic acid (PLA), polyglycolic acid (PGA) and mixtures and copolymers thereof, more preferably 50:50 copolymers of PLA:PGA and most preferably 75:25 copolymers of PLA:PGA. Single enantiomers of PLA may also be used, preferably L-PLA, either alone or in combination with PGA. Polycarbonates, polyfumarates and caprolactones may also be used to make the implants of this disclosure.

The amount of agent to be incorporated into the polymeric films of this disclosure is an amount effective to show a measurable effect in treating diseases having similar pathophysiological states, such as but not limited to heart failure. The composition of the present disclosure can be incorporated into the film by various techniques such as by solution methods, suspension methods, or melt pressing.

Transdermal delivery involves delivery of a therapeutic agent through the skin for distribution within the body by circulation of the blood. Transdermal delivery can be compared to continuous, controlled intravenous delivery of a drug using the skin as a port of entry instead of an intravenous needle. The therapeutic agent passes through the outer layers of the skin, diffuses into the capillaries or tiny blood vessels in the skin and then is transported into the main circulatory system.

Transdermal patch devices which provide a controlled, continuous administration of a therapeutic agent through the skin are well known in the art. Such devices, for example, are disclosed in U.S. Pat. Nos. 4,627,429; 4,784,857; 5,662,925; 5,788,983; and 6,113,940, which are all incorporated herein by reference. Characteristically, these devices contain a drug impermeable backing layer which defines the outer surface of the device and a permeable skin attaching membrane, such as an adhesive layer, sealed to the barrier layer in such a way as to create a reservoir between them in which the therapeutic agent is placed. In one embodiment of the present disclosure, a formulation of the agent is introduced into the reservoir of a transdermal patch and used by a patient who is homozygous for an insertional polymorphism at the PDE3A gene.

Another embodiment contemplates the incorporation of agent into a medical device that is then positioned to a desired target location within the body, whereupon the agent elutes from the medical device. As used herein, “medical device” refers to a device that is introduced temporarily or permanently into a mammal for the prophylaxis or therapy of a medical condition. These devices include any that are introduced subcutaneously, percutaneously or surgically to rest within an organ, tissue or lumen. Medical devices include, but are not limited to, stents, synthetic grafts, artificial heart valves, artificial hearts and fixtures to connect the prosthetic organ to the vascular circulation, venous valves, abdominal aortic aneurysm (AAA) grafts, inferior venal caval filters, catheters including permanent drug infusion catheters, embolic coils, embolic materials used in vascular embolization (e.g., PVA foams), mesh repair materials, a Dracon vascular particle orthopedic metallic plates, rods and screws and vascular sutures.

In one embodiment, the medical device such as a stent or graft is coated with a matrix. The matrix used to coat the stent or graft according to this disclosure may be prepared from a variety of materials. A primary requirement for the matrix is that it be sufficiently elastic and flexible to remain unruptured on the exposed surfaces of the stent or synthetic graft.

C. Dosages

The amount of drug that is administered or prescribed to the patient can be determined by a practitioner depending the clinical situation at hand. When provided in a discrete amount, each intake of an agent can be considered a “dose.” A medical practitioner may prescribe or administer multiple doses of an agent over a particular time course (treatment regimen) or indefinitely. Agents may be prescribed or administered 1, 2, 3, 4, 5, 6, 7, 8, 9, 10, 11, 12, 13, 14, 15, 16, 17, 18, 19, 20, 30, 40, 50, 60, 70, 80, or more times or any range derivable therein. It is further contemplated that the drug may be taken for an indefinite period of time or for as long as the patient exhibits symptoms of the medical condition for which the agent was prescribed or administered. Also, the drug may be administered every 2, 3, 4, 5, 6, 7, 8, 9, 10, 15, 20, 25, 30, 35, 40, 45, 50, 55 minutes, 1, 2, 3, 4, 5, 6, 7, 8, 9, 10, 11, 12, 13, 14, 15, 16, 17, 18, 19, 20, 21, 22, 23, 24 hours, or 1, 2, 3, 4, 5, 6, 7 days, or 1, 2, 3, 4, 5 weeks, or 1, 2, 3, 4, 5, 6, 7, 8, 9, 10, 11, 12 months or more, or any range derivable therein. Alternatively, it may be administered systemically over any such period of time and be extended beyond more than a year. Alternatively, a patient may be treated 1, 2, 3, 4, 5, or 6 times a day with the same or different doses. In some cases, the dose is up-titrated or down-titrated.

D. Combination Therapeutic Options

Multiple therapeutics can may be administered or taken at the same time. Examples of other therapies include, without limitation, beta blockers, anti-hypertensives, cardiotonics, anti-thrombotics, vasodilators, hormone antagonists, iontropes, diuretics, endothelin antagonists, calcium channel blockers, phosphodiesterase inhibitors, ACE inhibitors, angiotensin type 2 antagonists and cytokine blockers/inhibitors, and HDAC inhibitors.

Combinations may be achieved by administration of a single composition or pharmacological formulation that includes both agents, or by administration of two distinct compositions or formulations, at the same time. Alternatively, a therapy may precede or follow administration of another agent(s) by intervals ranging from minutes to weeks. In embodiments where the other therapies/agents are applied separately to the cell, one would generally ensure that a significant period of time did not expire between the time of each delivery, such that the agents would still be able to exert an advantageously combined effect on the subject. In such instances, it is contemplated that one would typically contact the cell with both modalities within about 12-24 hours of each other and, more preferably, within about 6-12 hours of each other, with a delay time of only about 12 hours being contemplated. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration will be desired. In this regard, various combinations may be employed. By way of illustration, where the first agent or treatment is “A” and the other agent or treatment is “B”, the following permutations based on 3 and 4 total administrations are exemplary:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are likewise contemplated.

1. Pharmacological Therapeutic Agents

Pharmacological therapeutic agents and methods of administration, dosages, etc., are well known to those of skill in the art (see for example, the “Physicians Desk Reference”, Klaassen's “The Pharmacological Basis of Therapeutics”, “Remington's Pharmaceutical Sciences”, and “The Merck Index, Eleventh Edition”, incorporated herein by reference in relevant parts), and may be combined with the disclosure in light of the disclosures herein. Some variation in dosage will necessarily occur depending on the condition of the subject being treated. The person responsible for administration will, in any event, determine the appropriate dose for the individual subject, and such individual determinations are within the skill of those of ordinary skill in the art.

Non-limiting examples of a pharmacological therapeutic agent that may be used in the present disclosure include an antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent, an antibacterial agent or a combination thereof.

a. Antihyperlipoproteinemics

In certain embodiments, administration of an agent that lowers the concentration of one of more blood lipids and/or lipoproteins, known herein as an “antihyperlipoproteinemic,” may be combined with a cardiovascular therapy according to the present disclosure, particularly in treatment of athersclerosis and thickenings or blockages of vascular tissues. In certain aspects, an antihyperlipoproteinemic agent may comprise an aryloxyalkanoic/fibric acid derivative, a resin/bile acid sequesterant, a HMG CoA reductase inhibitor, a nicotinic acid derivative, a thyroid hormone or thyroid hormone analog, a miscellaneous agent or a combination thereof.

Aryloxyalkanoic Acid/Fibric Acid Derivatives.

Non-limiting examples of aryloxyalkanoic/fibric acid derivatives include beclobrate, enzafibrate, binifibrate, ciprofibrate, clinofibrate, clofibrate (atromide-S), clofibric acid, etofibrate, fenofibrate, gemfibrozil (lobid), nicofibrate, pirifibrate, ronifibrate, simfibrate and theofibrate.

Resins/Bile Acid Sequesterants.

Non-limiting examples of resins/bile acid sequesterants include cholestyramine (cholybar, questran), colestipol (colestid) and polidexide.

HMG CoA Reductase Inhibitors.

Non-limiting examples of HMG CoA reductase inhibitors include lovastatin (mevacor), pravastatin (pravochol) or simvastatin (zocor).

Nicotinic Acid Derivatives.

Non-limiting examples of nicotinic acid derivatives include nicotinate, acepimox, niceritrol, nicoclonate, nicomol and oxiniacic acid.

Thyroid Hormones and Analogs.

Non-limiting examples of thyroid hormones and analogs thereof include etoroxate, thyropropic acid and thyroxine.

Miscellaneous Antihyperlipoproteinemics.

Non-limiting examples of miscellaneous antihyperlipoproteinemics include acifran, azacosterol, benfluorex, β-benzalbutyramide, carnitine, chondroitin sulfate, clomestrone, detaxtran, dextran sulfate sodium, 5,8,11,14,17-eicosapentaenoic acid, eritadenine, furazabol, meglutol, melinamide, mytatrienediol, ornithine, γ-oryzanol, pantethine, pentaerythritol tetraacetate, α-phenylbutyramide, pirozadil, probucol (lorelco), β-sitosterol, sultosilic acid-piperazine salt, tiadenol, triparanol and xenbucin.

b. Antiarteriosclerotics

Non-limiting examples of an antiarteriosclerotic include pyridinol carbamate.

c. Antithrombotic/Fibrinolytic Agents

In certain embodiments, administration of an agent that aids in the removal or prevention of blood clots may be combined with administration of a modulator, particularly in treatment of athersclerosis and vasculature (e.g., arterial) blockages. Non-limiting examples of antithrombotic and/or fibrinolytic agents include anticoagulants, anticoagulant antagonists, antiplatelet agents, thrombolytic agents, thrombolytic agent antagonists or combinations thereof

In certain aspects, antithrombotic agents that can be administered orally, such as, for example, aspirin and wafarin (coumadin), are contemplated.

Anticoagulants.

A non-limiting example of an anticoagulant include acenocoumarol, ancrod, anisindione, bromindione, clorindione, coumetarol, cyclocumarol, dextran sulfate sodium, dicumarol, diphenadione, ethyl biscoumacetate, ethylidene dicoumarol, fluindione, heparin, hirudin, lyapolate sodium, oxazidione, pentosan polysulfate, phenindione, phenprocoumon, phosvitin, picotamide, tioclomarol and warfarin.

Antiplatelet Agents.

Non-limiting examples of antiplatelet agents include aspirin, a dextran, dipyridamole (persantin), heparin, sulfinpyranone (anturane) and ticlopidine (ticlid).

Thrombolytic Agents.

Non-limiting examples of thrombolytic agents include tissue plaminogen activator (activase), plasmin, pro-urokinase, urokinase (abbokinase) streptokinase (streptase), anistreplase/APSAC (eminase).

d. Blood Coagulants

In certain embodiments wherein a patient is suffering from a hemmorage or an increased likelihood of hemmoraging, an agent that may enhance blood coagulation may be used. Non-limiting examples of a blood coagulation promoting agent include thrombolytic agent antagonists and anticoagulant antagonists.

Anticoagulant Antagonists.

Non-limiting examples of anticoagulant antagonists include protamine and vitamine K1.

Thrombolytic Agent Antagonists and Antithrombotics.

Non-limiting examples of thrombolytic agent antagonists include amiocaproic acid (amicar) and tranexamic acid (amstat). Non-limiting examples of antithrombotics include anagrelide, argatroban, cilstazol, daltroban, defibrotide, enoxaparin, fraxiparine, indobufen, lamoparan, ozagrel, picotamide, plafibride, tedelparin, ticlopidine and triflusal.

e. Antiarrhythmic Agents

Non-limiting examples of antiarrhythmic agents include Class I antiarrythmic agents (sodium channel blockers), Class II antiarrythmic agents (beta-adrenergic blockers), Class II antiarrythmic agents (repolarization prolonging drugs), Class IV antiarrhythmic agents (calcium channel blockers) and miscellaneous antiarrythmic agents.

Sodium Channel Blockers.

Non-limiting examples of sodium channel blockers include Class IA, Class IB and Class IC antiarrhythmic agents. Non-limiting examples of Class IA antiarrhythmic agents include disppyramide (norpace), procainamide (pronestyl) and quinidine (quinidex). Non-limiting examples of Class IB antiarrhythmic agents include lidocaine (xylocaine), tocainide (tonocard) and mexiletine (mexitil). Non-limiting examples of Class IC antiarrhythmic agents include encainide (enkaid) and flecamide (tambocor).

Beta Blockers.

Non-limiting examples of a beta blocker, otherwise known as a β-adrenergic blocker, a β-adrenergic antagonist or a Class II antiarrhythmic agent, include acebutolol (sectral), alprenolol, amosulalol, arotinolol, atenolol, befunolol, betaxolol, bevantolol, bisoprolol, bopindolol, bucumolol, bufetolol, bufuralol, bunitrolol, bupranolol, butidrine hydrochloride, butofilolol, carazolol, carteolol, carvedilol, celiprolol, cetamolol, cloranolol, dilevalol, epanolol, esmolol (brevibloc), indenolol, labetalol, levobunolol, mepindolol, metipranolol, metoprolol, moprolol, nadolol, nadoxolol, nifenalol, nipradilol, oxprenolol, penbutolol, pindolol, practolol, pronethalol, propanolol (inderal), sotalol (betapace), sulfinalol, talinolol, tertatolol, timolol, toliprolol and xibinolol. In certain aspects, the beta blocker comprises an aryloxypropanolamine derivative. Non-limiting examples of aryloxypropanolamine derivatives include acebutolol, alprenolol, arotinolol, atenolol, betaxolol, bevantolol, bisoprolol, bopindolol, bunitrolol, butofilolol, carazolol, carteolol, carvedilol, celiprolol, cetamolol, epanolol, indenolol, mepindolol, metipranolol, metoprolol, moprolol, nadolol, nipradilol, oxprenolol, penbutolol, pindolol, propanolol, talinolol, tertatolol, timolol and toliprolol.

Repolarization Prolonging Agents.

Non-limiting examples of an agent that prolong repolarization, also known as a Class III antiarrhythmic agent, include amiodarone (cordarone) and sotalol (betapace).

Calcium Channel Blockers/Antagonist.

Non-limiting examples of a calcium channel blocker, otherwise known as a Class IV antiarrythmic agent, include an arylalkylamine (e.g., bepridile, diltiazem, fendiline, gallopamil, prenylamine, terodiline, verapamil), a dihydropyridine derivative (felodipine, isradipine, nicardipine, nifedipine, nimodipine, nisoldipine, nitrendipine) a piperazinde derivative (e.g., cinnarizine, flunarizine, lidoflazine) or a miscellaneous calcium channel blocker such as bencyclane, etafenone, magnesium, mibefradil or perhexyline. In certain embodiments a calcium channel blocker comprises a long-acting dihydropyridine (nifedipine-type) calcium antagonist.

Miscellaneous Antiarrhythmic Agents.

Non-limiting examples of miscellaneous antiarrhymic agents include adenosine (adenocard), digoxin (lanoxin), acecamide, ajmaline, amoproxan, aprindine, bretylium tosylate, bunaftine, butobendine, capobenic acid, cifenline, disopyranide, hydroquinidine, indecamide, ipatropium bromide, lidocaine, lorajmine, lorcainide, meobentine, moricizine, pirmenol, prajmaline, propafenone, pyrinoline, quinidine polygalacturonate, quinidine sulfate and viquidil.

f. Antihypertensive Agents

Non-limiting examples of antihypertensive agents include sympatholytic, alpha/beta blockers, alpha blockers, anti-angiotensin II agents, beta blockers, calcium channel blockers, vasodilators and miscellaneous antihypertensives.

Alpha Blockers.

Non-limiting examples of an alpha blocker, also known as an α-adrenergic blocker or an α-adrenergic antagonist, include amosulalol, arotinolol, dapiprazole, doxazosin, ergoloid mesylates, fenspiride, indoramin, labetalol, nicergoline, prazosin, terazosin, tolazoline, trimazosin and yohimbine. In certain embodiments, an alpha blocker may comprise a quinazoline derivative. Non-limiting examples of quinazoline derivatives include alfuzosin, bunazosin, doxazosin, prazosin, terazosin and trimazosin.

Alpha/Beta Blockers.

In certain embodiments, an antihypertensive agent is both an alpha and beta adrenergic antagonist. Non-limiting examples of an alpha/beta blocker comprise labetalol (normodyne, trandate).

Anti-Angiotension II Agents.

Non-limiting examples of anti-angiotension II agents include angiotensin converting enzyme inhibitors and angiotension II receptor antagonists. Non-limiting examples of angiotension converting enzyme inhibitors (ACE inhibitors) include alacepril, enalapril (vasotec), captopril, cilazapril, delapril, enalaprilat, fosinopril, lisinopril, moveltopril, perindopril, quinapril and ramipril. Non-limiting examples of an angiotensin II receptor blocker, also known as an angiotension II receptor antagonist, an ANG receptor blocker or an ANG-II type-1 receptor blocker (ARBS), include angiocandesartan, eprosartan, irbesartan, losartan and valsartan.

Sympatholytics.

Non-limiting examples of a sympatholytic include a centrally acting sympatholytic or a peripherially acting sympatholytic. Non-limiting examples of a centrally acting sympatholytic, also known as an central nervous system (CNS) sympatholytic, include clonidine (catapres), guanabenz (wytensin) guanfacine (tenex) and methyldopa (aldomet). Non-limiting examples of a peripherally acting sympatholytic include a ganglion blocking agent, an adrenergic neuron blocking agent, a β-adrenergic blocking agent or a alpha1-adrenergic blocking agent. Non-limiting examples of a ganglion blocking agent include mecamylamine (inversine) and trimethaphan (arfonad). Non-limiting of an adrenergic neuron blocking agent include guanethidine (ismelin) and reserpine (serpasil). Non-limiting examples of a β-adrenergic blocker include acenitolol (sectral), atenolol (tenormin), betaxolol (kerlone), carteolol (cartrol), labetalol (normodyne, trandate), metoprolol (lopressor), nadanol (corgard), penbutolol (levatol), pindolol (visken), propranolol (inderal) and timolol (blocadren). Non-limiting examples of alpha1-adrenergic blocker include prazosin (minipress), doxazocin (cardura) and terazosin (hytrin).

Vasodilators.

In certain embodiments a cardiovasculator therapeutic agent may comprise a vasodilator (e.g., a cerebral vasodilator, a coronary vasodilator or a peripheral vasodilator). In certain particular embodiments, a vasodilator comprises a coronary vasodilator. Non-limiting examples of a coronary vasodilator include amotriphene, bendazol, benfurodil hemisuccinate, benziodarone, chloracizine, chromonar, clobenfurol, clonitrate, dilazep, dipyridamole, droprenilamine, efloxate, erythrityl tetranitrane, etafenone, fendiline, floredil, ganglefene, herestrol bis(β-diethylaminoethyl ether), hexobendine, itramin tosylate, khellin, lidoflanine, mannitol hexanitrane, medibazine, nicorglycerin, pentaerythritol tetranitrate, pentrinitrol, perhexyline, pimefylline, trapidil, tricromyl, trimetazidine, trolnitrate phosphate and visnadine.

In certain aspects, a vasodilator may comprise a chronic therapy vasodilator or a hypertensive emergency vasodilator. Non-limiting examples of a chronic therapy vasodilator include hydralazine (apresoline) and minoxidil (loniten). Non-limiting examples of a hypertensive emergency vasodilator include nitroprusside (nipride), diazoxide (hyperstat IV), hydralazine (apresoline), minoxidil (loniten) and verapamil.

Miscellaneous Antihypertensives.

Non-limiting examples of miscellaneous antihypertensives include ajmaline, γ-aminobutyric acid, bufeniode, cicletainine, ciclosidomine, a cryptenamine tannate, fenoldopam, flosequinan, ketanserin, mebutamate, mecamylamine, methyldopa, methyl 4-pyridyl ketone thiosemicarbazone, muzolimine, pargyline, pempidine, pinacidil, piperoxan, primaperone, a protoveratrine, raubasine, rescimetol, rilmenidene, saralasin, sodium nitrorusside, ticrynafen, trimethaphan camsylate, tyrosinase and urapidil.

In certain aspects, an antihypertensive may comprise an arylethanolamine derivative, a benzothiadiazine derivative, a N-carboxyalkyl(peptide/lactam) derivative, a dihydropyridine derivative, a guanidine derivative, a hydrazines/phthalazine, an imidazole derivative, a quanternary ammonium compound, a reserpine derivative or a sulfonamide derivative.

Arylethanolamine derivatives include amosulalol, bufuralol, dilevalol, labetalol, pronethalol, sotalol and sulfinalol. Benzothiadiazine derivatives include althizide, bendroflumethiazide, benzthiazide, benzylhydrochlorothiazide, buthiazide, chlorothiazide, chlorthalidone, cyclopenthiazide, cyclothiazide, diazoxide, epithiazide, ethiazide, fenquizone, hydrochlorothizide, hydroflumethizide, methyclothiazide, meticrane, metolazone, paraflutizide, polythizide, tetrachlormethiazide and trichlormethiazide.

N-Carboxyalkyl(Peptide/Lactam) derivatives include N-carboxyalkyl(peptide/lactam) alacepril, captopril, cilazapril, delapril, enalapril, enalaprilat, fosinopril, lisinopril, moveltipril, perindopril, quinapril and ramipril.

Dihydropyridine derivatives include amlodipine, felodipine, isradipine, nicardipine, nifedipine, nilvadipine, nisoldipine and nitrendipine.

Guanidine derivatives include bethanidine, debrisoquin, guanabenz, guanacline, guanadrel, guanazodine, guanethidine, guanfacine, guanochlor, guanoxabenz and guanoxan.

Hydrazines/Phthalazines include budralazine, cadralazine, dihydralazine, endralazine, hydracarbazine, hydralazine, pheniprazine, pildralazine and todralazine.

Imidazole derivatives include include clonidine, lofexidine, phentolamine, tiamenidine and tolonidine.

Quanternary Ammonium compounds include azamethonium bromide, chlorisondamine chloride, hexamethonium, pentacynium bis(methylsulfate), pentamethonium bromide, pentolinium tartrate, phenactropinium chloride and trimethidinium methosulfate.

Reserpine derivatives include bietaserpine, deserpidine, rescinnamine, reserpine and syrosingopine.

Suflonamide derivatives include ambuside, clopamide, furosemide, indapamide, quinethazone, tripamide and xipamide.

Vasopressors.

Vasopressors generally are used to increase blood pressure during shock, which may occur during a surgical procedure. Non-limiting examples of a vasopressor, also known as an antihypotensive, include amezinium methyl sulfate, angiotensin amide, dimetofrine, dopamine, etifelmin, etilefrin, gepefrine, metaraminol, midodrine, norepinephrine, pholedrine and synephrine.

g. Treatment Agents for Congestive Heart Failure

Non-limiting examples of agents for the treatment of congestive heart failure include anti-angiotension II agents, afterload-preload reduction treatment, diuretics and inotropic agents.

Afterload-Preload Reduction.

In certain embodiments, an animal patient that can not tolerate an angiotension antagonist may be treated with a combination therapy. Such therapy may combine administration of hydralazine (apresoline) and isosorbide dinitrate (isordil, sorbitrate).

Diuretics.

Non-limiting examples of a diuretic include a thiazide or benzothiadiazine derivative (e.g., althiazide, bendroflumethazide, benzthiazide, benzylhydrochlorothiazide, buthiazide, chlorothiazide, chlorothiazide, chlorthalidone, cyclopenthiazide, epithiazide, ethiazide, ethiazide, fenquizone, hydrochlorothiazide, hydroflumethiazide, methyclothiazide, meticrane, metolazone, paraflutizide, polythizide, tetrachloromethiazide, trichlormethiazide), an organomercurial (e.g., chlormerodrin, meralluride, mercamphamide, mercaptomerin sodium, mercumallylic acid, mercumatilin dodium, mercurous chloride, mersalyl), a pteridine (e.g., furtherene, triamterene), purines (e.g., acefylline, 7-morpholinomethyltheophylline, pamobrom, protheobromine, theobromine), steroids including aldosterone antagonists (e.g., canrenone, oleandrin, spironolactone), a sulfonamide derivative (e.g., acetazolamide, ambuside, azosemide, bumetanide, butazolamide, chloraminophenamide, clofenamide, clopamide, clorexolone, diphenylmethane-4,4′-disulfonamide, disulfamide, ethoxzolamide, furosemide, indapamide, mefruside, methazolamide, piretanide, quinethazone, torasemide, tripamide, xipamide), a uracil (e.g., aminometradine, amisometradine), a potassium sparing antagonist (e.g., amiloride, triamterene) or a miscellaneous diuretic such as aminozine, arbutin, chlorazanil, ethacrynic acid, etozolin, hydracarbazine, isosorbide, mannitol, metochalcone, muzolimine, perhexyline, ticrnafen and urea.

Inotropic Agents.

Non-limiting examples of a positive inotropic agent, also known as a cardiotonic, include acefylline, an acetyldigitoxin, 2-amino-4-picoline, aminone, benfurodil hemisuccinate, bucladesine, cerberosine, camphotamide, convallatoxin, cymarin, denopamine, deslanoside, digitalin, digitalis, digitoxin, digoxin, dobutamine, dopamine, dopexamine, enoximone, erythrophleine, fenalcomine, gitalin, gitoxin, glycocyamine, heptaminol, hydrastinine, ibopamine, a lanatoside, metamivam, milrinone, nerifolin, oleandrin, ouabain, oxyfedrine, prenalterol, proscillaridine, resibufogenin, scillaren, scillarenin, strphanthin, sulmazole, theobromine and xamoterol.

In particular aspects, an intropic agent is a cardiac glycoside, a beta-adrenergic agonist or a phosphodiesterase inhibitor. Non-limiting examples of a cardiac glycoside includes digoxin (lanoxin) and digitoxin (crystodigin). Non-limiting examples of a β-adrenergic agonist include albuterol, bambuterol, bitolterol, carbuterol, clenbuterol, clorprenaline, denopamine, dioxethedrine, dobutamine (dobutrex), dopamine (intropin), dopexamine, ephedrine, etafedrine, ethylnorepinephrine, fenoterol, formoterol, hexoprenaline, ibopamine, isoetharine, isoproterenol, mabuterol, metaproterenol, methoxyphenamine, oxyfedrine, pirbuterol, procaterol, protokylol, reproterol, rimiterol, ritodrine, soterenol, terbutaline, tretoquinol, tulobuterol and xamoterol. Non-limiting examples of a phosphodiesterase inhibitor include enoximone and aminone (inocor).

Antianginal Agents.

Antianginal agents may comprise organonitrates, calcium channel blockers, beta blockers and combinations thereof. Non-limiting examples of organonitrates, also known as nitrovasodilators, include nitroglycerin (nitro-bid, nitrostat), isosorbide dinitrate (isordil, sorbitrate) and amyl nitrate (aspirol, vaporole).

2. Surgical Intervention

In certain aspects, the secondary therapeutic agent may comprise a surgery of some type, which includes, for example, preventative, diagnostic or staging, curative and palliative surgery. Surgery, and in particular a curative surgery, may be used in conjunction with other therapies, such as the present disclosure and one or more other agents.

Such surgical interventions for vascular and cardiovascular diseases and disorders are well known to those of skill in the art, and may comprise, but are not limited to, performing surgery on an organism, providing a cardiovascular mechanical prostheses, angioplasty, coronary artery reperfusion, catheter ablation, providing an implantable cardioverter defibrillator to the subject, mechanical circulatory support or a combination thereof. Non-limiting examples of a mechanical circulatory support that may be used in the present disclosure comprise an intra-aortic balloon counterpulsation, left ventricular assist device or combination thereof.

### IV. KITS

In some embodiments, the present disclosure provides kits for the detection of PDE3A polymorphisms. In some embodiments, the kits contain reagents specific for the detection or analysis of DNA (e.g., oligonucleotide probes or primers). In particular embodiments, the kits contain all of the components necessary to perform a detection assay, including controls. In some embodiments, individual probes and reagents for detection of PDE3A polymorphisms are provided as analyte specific reagents. A kit can also include, for example, a PDE3A assay such as an ELISA. In addition, a kit can contain control samples, for example, to show that amplification reactions are not contaminated. In other embodiments, the kits are provided as in vitro diagnostics. The contents of the kit are contained in packaging material, preferably to provide a sterile, contaminant-free environment.

In addition, the packaging material contains instructions indicating how the materials within the kit can be employed. The instructions for use typically include a tangible expression describing the reagent concentration or at least one assay method parameter, such as the relative amounts of reagent and sample to be admixed, maintenance time periods for reagent/sample admixtures, temperature, buffer conditions, and the like. Software for analysis and presentation of results may also be included.

### V. EXAMPLES

The following examples are given for the purpose of illustrating various embodiments of the disclosure and are not meant to limit the present disclosure in any fashion. One skilled in the art will appreciate readily that the present disclosure is well adapted to carry out the objects and obtain the ends and advantages mentioned, as well as those objects, ends and advantages inherent herein. The present examples, along with the methods described herein are presently representative of particular embodiments, are exemplary, and are not intended as limitations on the scope of the disclosure. Changes therein and other uses which are encompassed within the spirit of the disclosure as defined by the scope of the claims will occur to those skilled in the art.

### Example 1

### Materials and Methods

Tissue Procurement.

Human subjects with end-stage heart failure were males and females of all ages, races and ethnic backgrounds with end-stage heart failure who gave written consent to donate their hearts to the institutional review board-approved cardiac transplant tissue bank at the University of Colorado. Non-failing hearts that could not be used for transplant were obtained from organ donors, with consent for research use given by family members. Explanted hearts used for genotyping were from 315 heart failure patients who exhibited severe left ventricular (LV) dysfunction (LVEF in %=20.6±9.1, Table 1) compared to the 110 non-failing (NF) control hearts (LVEF %=59.5±12.9). A subset of hearts that did not differ in baseline characteristics from the genotyping cohort was used for molecular and biochemical studies (Table 1). Seventy three of the 173 failing hearts in this subset were treated pretransplant with the PDE3 inhibitors milrinone (administered intravenously at the therapeutic doses of 0.25-0.75 ng/Kg) or enoximone (administered orally at 25-75 mg t.i.d.; Lee et al., 1991) for hemodynamic stabilization as a bridge-to-transplant. The baseline characteristics of the two groups of failing hearts did not differ (Table 1).

DNA Extraction and Genotyping.

Genomic DNA was isolated using a modification of Chomczynski's method (Chomczynski et al., 1987). Briefly, frozen tissue was digested with proteinase K followed by phenol:chloroform extraction and isopropanol precipitation. PCR was performed using the BioReady Taq DNA polymerase (Bulldog). The resulting PCR product was separated in a 3% agarose gel. The following primers were used for genotype purposes:

PDE3A F:
5′ CCACTGCCATTGACTAGCTG
PDE3A R:
5′ GCCAAAAGGAGATCCTTGAGAT

mRNA extraction and RT-PCR.

RNA was extracted from left ventricular mid free wall 1 g aliquots of 44 NF and 98 nonischemic (NICM) or ischemic cardiomyopathy (ISCM) failing adult hearts. Tissue aliquots from ISCM hearts used for RNA extraction or biochemical measurements were from infarct-free areas. mRNA extraction was performed using the mirVana™ kit (Ambion) according to manufacturer's recommendation. RT-PCR was performed as previously described (Sucharov et al., 2008) using PDE3A1 specific primers:

PDE3A1 F:
5′ GCTCCGGAGCTCTCGGAAA
PDE3A1 R:
5′ CCAGCAGCGCCAGCAGAAA

cAMP Assay.

ELISA was performed by the core facility at Children's Hospital Colorado, Aurora, Colo. using the R&D Parameter immunoassay kit (R&D Systems, Minneapolis, Minn.) according to manufacturer's recommendations, in homogenates of 100 mg aliquots of left ventricular mid-free wall myocardium that had been immediately frozen at −80° C. on cardiac explantation.

β-Adrenergic Receptor Density.

Total β (β₁, β₂ and β₃)-adrenergic receptor (AR) density was measured in crude LV membranes by ¹²⁵[I]CYP binding, as previously described (Lowes et al., 1997).

NRVM Preparation.

Neonatal rat ventricular myocytes (NRVMs) were prepared from the hearts of 2-day-old rat pups by enzymatic digestion as described (Simpson et al., 1982; Waspe et al., 1990; Singh et al., 2000).

Construct Design and Cloning.

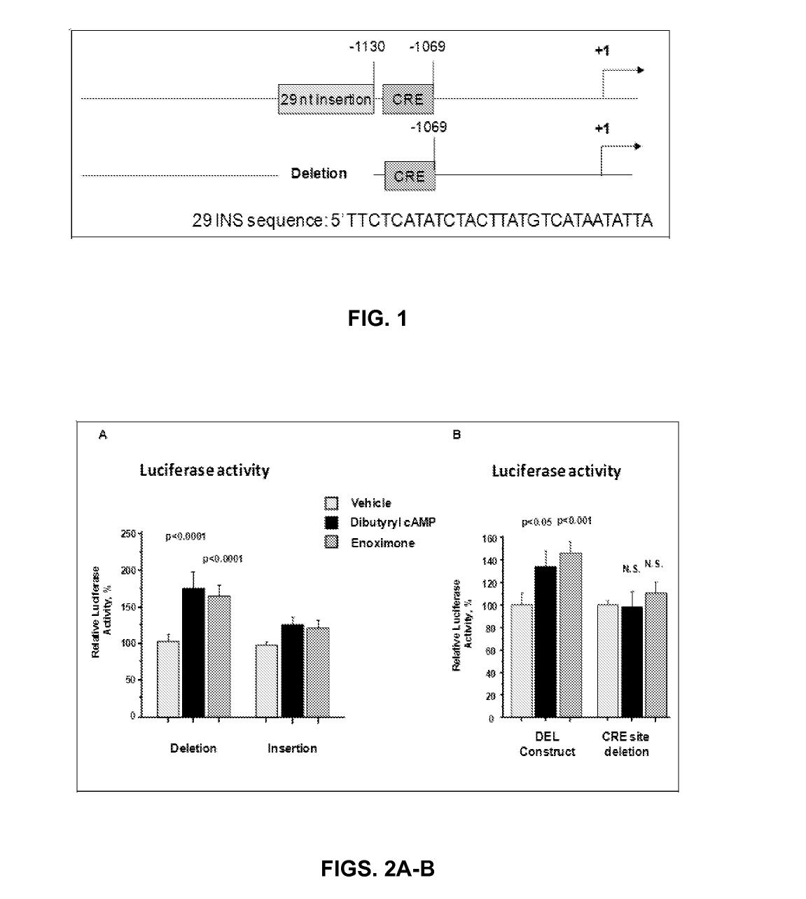

Cloning of the PDE3A promoter region was performed using PCR primers that amplified the −1434/+440 region of the human promoter that contains the INS/DEL genotype. The resulting fragment was cloned in the Promega pGL3-basic vector using MluI and BglII sites as previously described (Sucharov et al., 2003). Cloning of the construct that lacks the cAMP response element (CRE) was done by amplifying the −1066/+440 region of the PDE3A promoter downstream from the CRE site. A schematic description of the promoter is presented in FIG. 1.

Transfection and Luciferase Activity Assays.

Firefly luciferase plasmid DNA was transfected using the Lonza system as previously described (Sucharov et al., 2008). Twenty-four hours after transfection cells were treated with 1 μμmol/L cAMP or 5 μmol/L enoximone for 48 hours. Luciferase activity was determined as previously described (Sucharov et al., 2003).

Preparation of Subcellular Fractions from Human Left Ventricular Myocardium.

Approximately 150 mg of −80° C. frozen left ventricular mid free wall myocardium as described above for cAMP assays was homogenized and separated into nuclear, cytosolic, and sarcoplasmic reticulum enriched microsomal fractions by differential sedimentation by a protocol adapted from previously published methods (Smith et al., 1993). Tissue was homogenized using a polytron for two 5-second cycles in 5 volumes of 0.29 mol/L sucrose, 10 mmol/L 3-[N-morpholino]propanesulfonic acid, 1 mmol/L benzamidine, 2 mmol/L EGTA, phosphatase inhibitor, and protease inhibitor (“sucrose buffer”). The homogenate was sedimented at 7,700 g at 4° C. for 30 minutes. The pellet was resuspended in 4 volumes of sucrose buffer without EGTA, and designated the “nuclear” fraction. The supernatant was resedimented at 50,000 rpm at 4° C. for 60 minutes. The supernatant was designated the “cytosolic” fraction. The pellet was resuspended in 1 volume of sucrose buffer without EGTA and was designated the “microsomal” fraction.

PDE3 Activity Assay.

cAMP-hydrolytic activity was quantified according to the method of Kincaid and Manganiello (1988) as described in detail previously (Movesian et al., 1991). Briefly, microsomal fractions were added to a reaction mixture containing 0.3 mol/L NaHEPES, 0.6 mmol/L EGTA, 1 mol/L MgCl₂ (pH 7.5) and 0.1 μmol/L [³H]cAMP (Perkin Elmer, Boston, Mass.). cAMP hydrolysis occurred for 10 minutes at 30° C., then the reaction was stopped by the addition of 10.1 mmol/L unlabeled cAMP and 5 mol/L unlabeled 5′AMP in 0.25 mol/L HCl. The reaction mixture was neutralized with NaOH. [³H]5′AMP was converted to [³H]adenosine by incubation with Crotalus atrox venom (30 mg/ml) for 30 minutes at 30° C. The reaction mixture was then applied to QAE-Sephadex columns, and [³H]adenosine was eluted with water. Cyclic AMP hydrolysis in pmol/min/mg was determined by measuring [³H]adenosine in the eluent. PDE activity was measured in the presence and absence of 0.1 μmol/L cilostazol, a concentration that inhibits rtPDE3A submaximally (IC₄₄); PDE3 activity was calculated by dividing the difference in activity in the presence and absence of cilostazol by the fractional inhibition of PDE3 activity at this concentration.

Statistical Analysis:

Statistical analyses were performed using Statview software (SAS Institute, Cary, N.C.). Statistical significance was set a priori at p<0.05 and all data are presented as mean±SEM. Analyses of data with >2 groups was by ANOVA with a Scheffe multiple comparison test, between two groups was by t-test, and categorical variables and Hardy-Weinberg equilibrium were analyzed by the Chi-square test.

### Example 2

### Results

Identification of an Insertion/Deletion Polymorphism in the Promoter Region of the PDE3A Gene.

In order to determine if there are polymorphisms in the PDE3A promoter region that could affect mRNA expression and response to PDE3 inhibitor treatment, PDE3A1 cDNA including the upstream 10 Kb promoter region was sequenced in 60 samples of failing left ventricular myocardium from the explanted hearts of transplant recipients that were outliers in a PDE3A1 mRNA expression screen.

As shown in FIG. 1, sequencing of the promoter region resulted in the identification of a 29-nucleotide deletion (DEL)/insertion (INS) polymorphism beginning at −1130. The presence of the INS/DEL polymorphism was subsequently analyzed in 425 left ventricular tissue samples from a human heart tissue bank (Table 1). For the 110 non-failing hearts examined; 33.6% were DEL homozygotes, 18.2% were INS homozygotes and 48.2% were heterozygotes (HET), for respective DEL and INS allele frequencies of 0.58 and 0.42. The genotype frequencies in non-failing hearts were in Hardy-Weinberg equilibrium (Chi square=0.89). Analysis of 315 failing hearts with severe LV dysfunction showed that 44.5% were DEL homozygotes, 20.3% were INS homozygotes and 35.2% were HET, for respective DEL and INS allele frequencies of 0.62 and 0.38 (p=0.49 vs. non-failing). Comparison of genotype distribution by Chi-square between non-failing and failing hearts showed differences between the two groups (p<0.0001), and failing heart genotype frequencies were not in Hardy-Weinberg equilibrium (p<0.0001). Of the 425 subjects, 17 were African-American with the following genotype distribution: 70.6% DEL homozygotes and 29.4% HET, for respective DEL and INS allele frequencies of 0.85 and 0.15. The remaining “non African-American” genotype frequencies were 40% DEL homozygotes, 21% INS homozygotes and 39% HET (p=0.02 vs. African-American).

Polymorphism Effects on PDE3A Promoter Activity.

To determine the effect of the INS/DEL polymorphism on PDE3A expression in response to agents that act through cAMP, the promoter region containing the INS or DEL polymorphism was cloned in the PGL3 basic vector and transfected into NRVMs treated with dibutyryl cAMP or the PDE3 inhibitor enoximone. As shown in FIG. 2A, treatment of the cells with dibutyryl cAMP or enoximone resulted in increased luciferase activity of the DEL promoter construct, whereas the INS construct exhibited no evidence of up-regulation of PDE3A expression. Analysis of the INS region showed potential binding sites for the transcription factors GATA4 and EVI-1, but electrophoretic mobility assays using GATA4 antibody or EVI-1 in vitro translated protein failed to show interaction of these transcription factors with the INS region (data not shown).

A cAMP Response Element Mediates Increased Promoter Activity in DEL.

Higher luciferase activity of the DEL promoter construct in response to increased cAMP levels suggested that the PDE3A promoter contains a cAMP-response element (CRE) that activates PDE3A expression in the presence of cAMP. Further analysis of the PDE3A promoter region revealed the presence of a CRE (-5′TAAGTCATCT) 51 bp downstream from the INS region. The PDE3A DEL construct containing the CRE resulted in increased luciferase activity in response to cAMP or enoximone, while deletion of the CRE sequence prevented cAMP-induced increases in promoter activity (FIG. 2B).

Myocardial PDE3A1 mRNA Abundance is Increased in the Presence of the DEL Polymorphism.

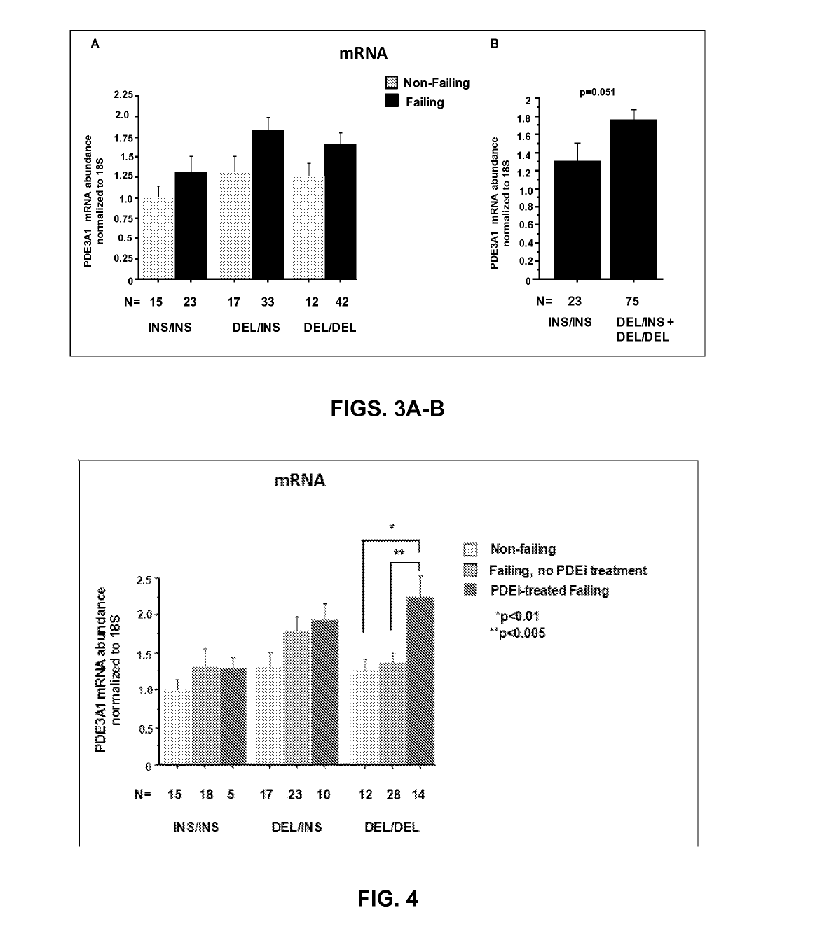

Demographic/other characteristics of hearts used in gene expression and biochemical experiments are given in Table 1. In order to determine the effect of the polymorphic region on left ventricular PDE3A1 mRNA expression, RT-PCR was performed in non-failing and failing human hearts regardless of their exposure to PDE3 inhibitors (FIG. 3A). By ANOVA across the three genotypes there were no statistically significant differences in mRNA expression between non-failing and failing hearts, but values tended to be higher in the failing groups. Within the failing hearts, there was a trend for increased (by 35%, p=0.051) PDE3A mRNA expression in the two genotypes containing DEL alleles compared to INS homozygotes (FIG. 3B).

PDE3 Inhibitor Treatment is Associated with Increased PDE3A1 mRNA Abundance in the Failing Hearts of Patients with the DEL Promoter Polymorphism.

In order to determine if treatment of heart failure patients with PDE3 inhibition affects PDE3A 1 mRNA levels, PDE3A expression was analyzed in failing hearts from patients who had (n=69) or hadn't (n=29) been treated with PDE3 inhibitors and compared to 44 non-failing controls. There were no differences in baseline characteristics including LVEF between PDE inhibitor treated and untreated patients (Table 1). As shown in FIG. 4, in DEL homozygotes prior PDE3 inhibition in failing hearts was associated with an increased PDE3A1 mRNA expression compared to non-failing (by 1.8 fold, p=0.037) or failing (by 1.7 fold, p=0.024) hearts not treated with PDE3 inhibitors. In contrast, heterozygotes or INS homozygotes treated with PDE inhibitors did not exhibit up-regulation in PDE3A1 mRNA abundance.

PDE3 Inhibitor Treatment is Associated with Increased PDE3 Enzyme Activity in DEL Homozygous Failing Hearts.

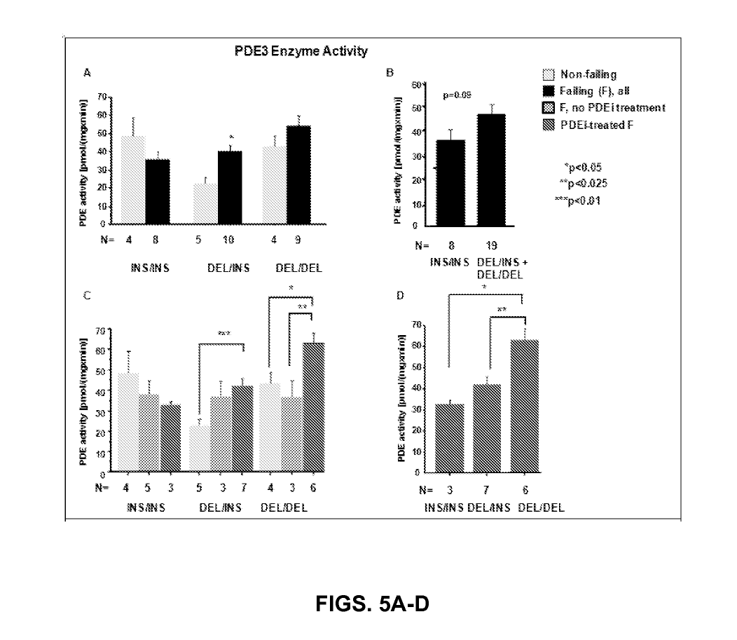

Compared to the 13 non-failing hearts PDE3 activity tended to be increased in the failing LVs of heterozygotes (ANOVA p=0.007, Scheffe p=0.39, t-test p=0.007) (FIG. 5A). As for mRNA levels, in failing hearts there was a trend for increased (by 30%, p=0.09) PDE3 enzyme activity in the two genotypes containing DEL alleles compared to INS homozygotes (FIG. 5B). In the LVs of the 16 patients treated with PDE3 inhibitors, enzyme activity was increased in heterozygotes and DEL homozygotes when compared to non-failing hearts (FIG. 5C). In addition, myocardial PDE3 activity was increased in DEL homozygous patients treated with PDE3 inhibitors when compared to the 11 heart failure patients not treated with PDE3 inhibitors (FIG. 5C). FIG. 5D shows PDE3 enzyme activity from failing hearts from patients treated with PDE3 inhibitors. DEL homozygotes had a 1.9 fold higher (p=0.004) myocardial PDE3 enzyme activity than INS homozygotes, and a 1.5 fold higher (p=0.01) activity than heterozygotes. Although heterozygotes tended to have higher PDE3 activities than INS homozygotes, the 1.3-fold difference was not statistically significant (p=0.48). There were no differences in myocardial PDE3 activity for the different genotypes in heart failure patients not treated with PDE3 inhibitors (FIG. 5C).

cAMP Levels are Lower in HF Patients Lacking the INS Polymorphism.

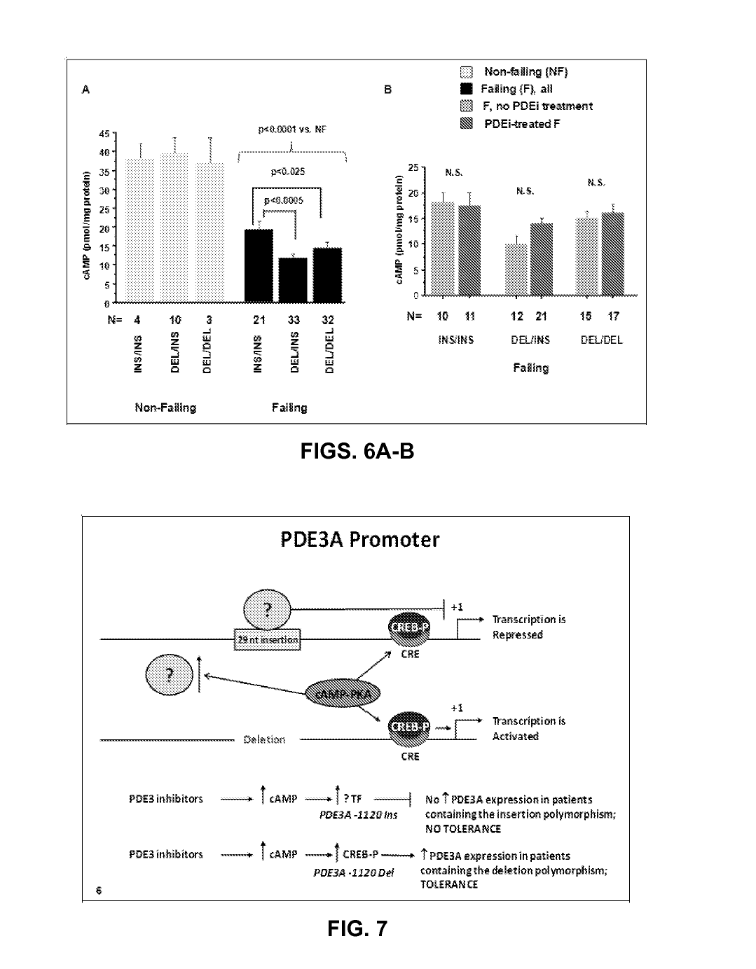

The inventors asked if the INS/DEL polymorphism might affect myocardial tissue cAMP levels. As shown in FIG. 6A, and as previously reported by others (Danielsen et al., 1989; von der Leyen et al., 1991; Bohm et al., 1994), cAMP levels are decreased in failing human hearts. Compared to INS homozygotes this decrease is statistically significantly accentuated in both heterozygotes (by 40%) and DEL homozygotes (by 24%), regardless of exposure to PDE3 inhibitors (FIG. 6B).

β-AR Receptor Density.

Total β-AR density was decreased in 63 failing LVs (Bₘₐₓ=58.1±26.5 fmol/mg compared to 81.7±23.3 fmol/mg in 22 non-failing, p=0.0004). Within the failing group, there was no difference in LV β-AR density between 23 patients treated with PDE inhibitors (55.8±28.3 fmol/mg) versus 40 untreated patients (59.4±25.7 fmol/mg, p=0.61). There were no differences among the three failing genotype groups (n=14 INS homozygotes, 53.4±17.8 fmol/mg; 22 heterozygotes, 66.4±34.1 fmol/mg; 27 DEL homozygotes 53.8±22.0 fmol/mg; ANOVA p=0.19), and no difference between INS homozygotes and DEL carriers (59.4±28.5 fmol/mg, p=0.43).

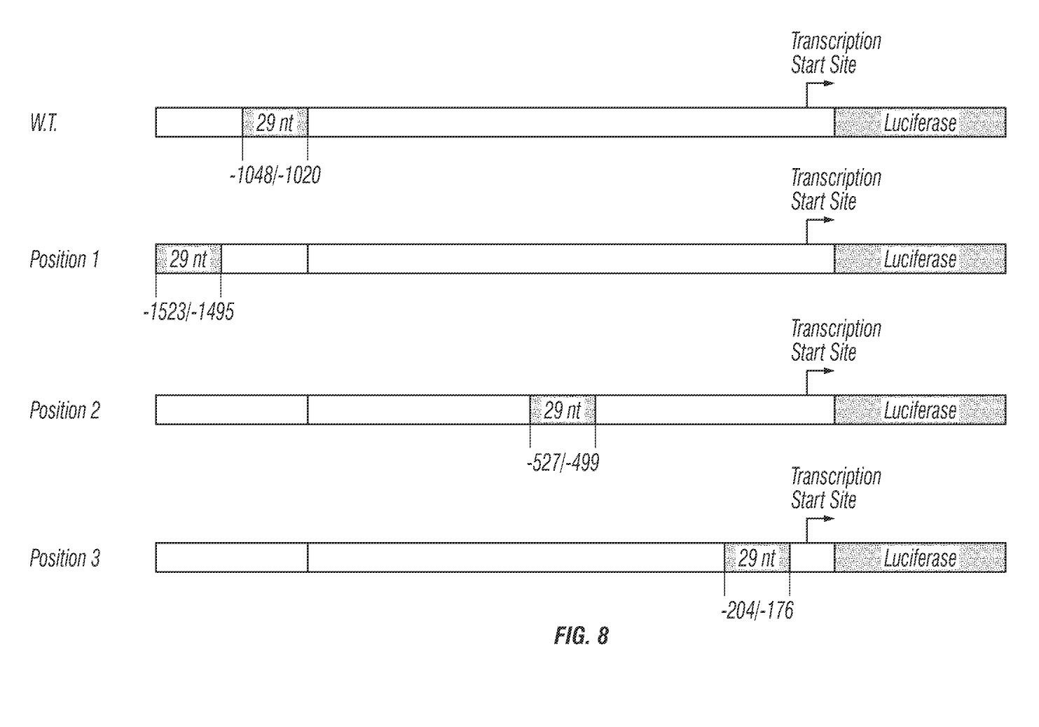

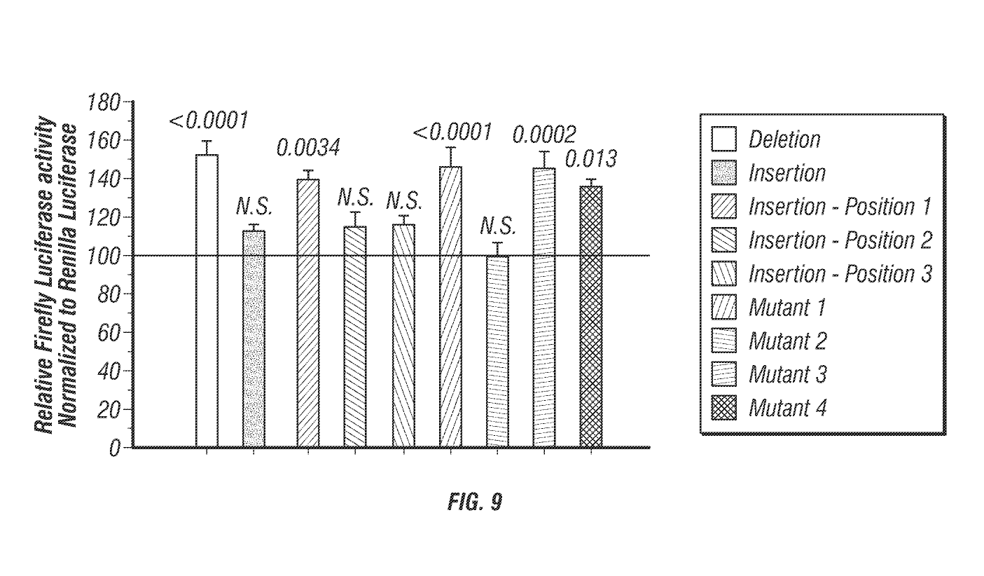

Since the factor that binds to the 29 nucleotide Insertion Region is not known, the inventors attempted to determine if the effect of the Insertion Region in regulating the PDE3 promoter was affected by the position of the Insertion Region, therefore altering DNA bending. It is also possible that the specific sequence is important for recognition by a transcription factor. To determine if the position of the Insertion Region affects its ability to prevent cAMP-mediated increase in the PDE3 promoter activity, the 29 nucleotide sequence was cloned in different positions in the promoter as shown in FIG. 8. To determine if mutating the 29 nucleotide sequence would have an effect on promoter activity, random mutations were generated and cloned in the same position as the wild-type construct (Table 3). Neonatal rat ventricular myocytes (NRVMs) were transfected with the Del and INS constructs, constructs where the position of the INS sequence was altered (Positions 1, 2 and 3), and 4 mutant constructs (Mutant 1-4). Cells were treated with cAMP 24 hours after transfection, harvested 48 hours after treatment, and luciferase activity was measured. As shown in FIG. 9, moving the INS upstream from its original position (Position 1), did affect its ability to prevent cAMP-mediated increase in promoter activity. Positions 2 and 3 behaved as the wild-type. Three of the mutants (Mutants 1, 3 and 4) lost the ability to prevent cAMP-mediated increase in promoter activity. These results suggest that a transcription factor likely recognizes and binds to the 29 nucleotide INS, repressing cAMP-mediated increase in PDE3A levels. It is interesting that moving the wild-type sequence upstream from its original position prevents its function. This may be due to interaction with other factors that are in proximity to the INS region.

TABLE 1
Baseline characteristics, demographics of study subjects

| | | | | | |
|---|---|---|---|---|---|
| Type | AGE | Ethnicity | Gender | LVEF (%) | Etiology |

Genotyping

| | | | | | |
|---|---|---|---|---|---|
| NF | 47 ± 13.3 | 82.7% - W | 53.6% - M | 59.5 ± 12.9 | NA |
| (n = 110) | | 12.7% - H | | | |
| | | 3.7% - AA | | | |
| | | 0.9% - NA | | | |
| F | 49.6 ± 13 | 59.2% W | 79.1% - M | 20.6 ± 9.1 | 63% NICM |
| (n = 315) | | 5.0% - H | | | 37% ISCM |
| | | 4.4% - AA | | | |
| | | 0.95% - NA | | | |
| | | 0.6% - A | | | |
| | | 0.6% - ME | | | |
| | | 29.05% - U | | | |

Molecular/biochemical data

| | | | | | |
|---|---|---|---|---|---|
| NF | 46.85 ± 12.76 | 78.37% - W | 52.7% - M | 61.4 ± 12.5 | NA |
| (n = 74) | | 17.57% - H | | | |
| | | 4.05% - AA | | | |
| F, PDEi | 51.1 ± 13.2 | 48.7% - W | 80.0% - M | 18.2 ± 9.3 | 65.8% NICM |
| (n = 73) | | 5.3% - AA | | | 34.2% ISCM |
| | | 1.3% - H | | | |
| | | 1.3% - ME | | | |
| | | 43.4% - U | | | |
| F, no PDEi | 49.8 ± 12.7 | 69% - W | 84.2% - M | 17.8 ± 9.0 | 55% NICM |
| (n = 100) | | 4% - H | | | 44% ISCM |
| | | 3% - AA | | | |
| | | 3% - NA | | | |
| | | 1% - A | | | |
| | | 1% - ME | | | |
| | | 19% - U | | | |

NF—Nonfailing; F—Failing; PDEi = Type 3 phosphodiesterase inhibitor treatment; W—White; H—Hispanic; AA—African American; A—Asian; ME—Middle-Eastern; U—Unknown; NA—Native American M—Male; LVEF—Left Ventricular Ejection Fraction; NICM = nonischemic cardiomyopathy; ISCM = ischemic cardiomyopathy.

TABLE 2
Non-Standard Abbreviations

| Abbreviation/ | |
|---|---|
| acronym | Definition |
| PDE3A | Phosphodiesterase 3A gene |
| PDE3A | Phosphodiesterase 3A mRNA, protein or |
| | enzyme activity |
| PDE3A1 | PDA3A1 protein or mRNA, 136,000 Mr |
| | longest isoform of PDE3 |
| INS | Insertion |
| DEL | Deletion |
| LV | Left ventricle |
| β-ARs | Beta-adrenergic receptors |
| Bmax | Maximum displaceable binding |
| AC | Adenylyl cyclase |
| PKA | Protein kinase A |
| CaMKII | Ca²⁺/calmodulin-dependent protein kinase II |
| PDEs | Cyclic nucleotide phosphodiesterases |
| HF | Heart failure |
| LTCC | L-type Ca²⁺ channels |
| SR | Sarcoplasmic reticulum |
| SERCA2 | Sarcoplasmic reticulum ATPase 2 |
| PLN | Phospholamban |
| RyR2 | Ryanodine receptor 2 |
| NRVMs | Neonatal rat ventricular myocytes |

TABLE 3-29
Nucleotide wild-type and mutant sequences

| | |
|---|---|
| Wild Type | TTCTCATATCTACTTATGTCATAATATTA |
| Mutant 1 | CCTCTGCGCTCGTCCGCACTGCGGCGCCG |
| Mutant 2 | GAGCATTCGTAGCCTAGTAGTCCGTAGAT |
| Mutant 3 | ACATTCGTACCAGTGGACCACTTAGGACC |
| Mutant 4 | TGCCGAAGTTGCACATTGGCACGCATTGA |

### Example 3

### Discussion

Treatment with PDE3 inhibitors has been evaluated in multiple clinical trials for acute and chronic heart failure, and is currently used as a bridge to transplant or in weaning from cardiopulmonary by-pass. Although an initial response is typically hemodynamically beneficial, long-term treatment may lead to adverse effects that include arrhythmias and sudden cardiac death. Furthermore, administration of PDE3 inhibitors can result in tolerance that can be observed as early as 72 hours into continuous infusion (Maisel et al., 1989; Packer et al., 1984; Maskin et al., 1982). Continuous intravenous treatment of heart failure patients with the PDE3 inhibitor amrinone results in an increase in cardiac output at 24 hours, with a return to baseline values by 72 hours (Maisel et al., 1989). Down-regulation and desensitization of the β-ARs was observed in lymphocytes from these treated patients, but in another study (Lee et al., 1991) chronic PDE3 inhibitor treatment with enoximone did not affect myocardial β-AR density.

In the current study, the inventors identified a 29-nucleotide INS/DEL polymorphism in the promoter region that was associated with increased PDE3A1 mRNA levels in PDE3 inhibitor-treated patients who were DEL homozygous. In patients who had DEL genotypes, PDE3 inhibitor treatment resulted in gene-dose related increased PDE3 enzyme activity in severely failing human LVs, providing an alternative molecular mechanism for tolerance to PDE3 inhibitors in a subset of patients. Furthermore, frequency analysis of this polymorphic variation showed a disproportionate increase in DEL homozygotes in the failing population, and DEL genotypes were associated with greater degrees of cAMP down-regulation in failing LVs. In addition, the INS homozygote polymorphism was not detected in African-American subjects, suggesting a possible racial effect on distribution of the INS/DEL polymorphism. Thus the −1130 INS/DEL polymorphism in the PDE3A promoter has the potential to affect treatment response to PDE3 inhibitors, as well as the natural history of heart failure.

As schematized in FIG. 7, deletion of the −1130 insertion results in the “unmasking” of a CRE downstream from the polymorphic region. In cultured cardiac myocytes transfected with a PDE3A DEL promoter-luciferase reporter construct, increased cAMP levels resulted in promoter activation through the CRE binding site, and in failing human hearts with at least one copy of the PDE3A DEL polymorphism treatment with PDE3 inhibitors was associated with higher PDE3A1 mRNA levels. For PDE3 enzyme activity, the DEL allele conferred a gene-dose effect, with heterozygotes being intermediate between INS homozygotes and DEL homozygotes. The induction of PDE3A gene expression by a cAMP positive feedback loop would blunt the effect of PDE inhibitor treatment by increasing the expression of the drug-targeted enzyme. Such cAMP-induced up-regulation in PDE expression resulting in PDE inhibitor tolerance has been observed for other phosphodiesterases (Compos-Toimil et al., 2008; Lin et al., 2003), and cAMP-induced up-regulation in PDE3A gene expression has been reported in porcine ovarian cumulus cells (Sasserville et al., 2007). In the human PDE3A gene the −1069 CRE mediates cAMP-PKA-CREB up-regulation of gene transcription and expression, which is prevented by the repressor INS polymorphism upstream from the CRE.

Whether a transcription factor binds to the 29-nucleotide region of the promoter is unclear. The inventors performed electrophoretic mobility-shift assays in an attempt to identify the factor that could prevent activation of the CRE site, but were unable to detect a specific transcription factor interacting with that region. It is possible that deletion of this region results in a conformational change that either favors binding to the CRE site, or that the 29-nucleotide insertion results in a conformational change that favors interaction with another transcription factor or a regulatory noncoding RNA that represses or prevents protein interaction with the CRE. Further studies will be needed to define the mechanism of repression/activation by the 29-nucleotide polymorphism.

Because the SR SERCA2-PLN microdomain is the likely site of the inotropic/lusitropic effects of PDE3 inhibitors (Movesian et al., 2009; Beca et al., 2013; Lowes et al., 2005) the inventors measured PDE3 activity in sarcoplasmic reticulum (SR)-enriched microsomes, as well as mRNA expression of the transcript encoding for the PDE3A1 isoform that is restricted to microsomal fractions (Movesian et al., 2009; Wechsler et al., 2002; Hambleton et al., 2005). In LV free wall aliquots of failing human hearts, the inventors found that microsomal fraction PDE3 enzyme activity and mRNA were unchanged, or in PDE3 inhibitor-treated DEL homozygotes, increased. Previous studies have found that PDE3 enzyme activity was either unchanged (Movesian et al., 1991; von der Leyen et al., 1991) or decreased (Ding et al., 2005) in failing human LVs, and in the latter study PDE3A protein abundance was also decreased (Ding et al., 2005). One previous study reporting unchanged PDE3 enzyme activity in failing hearts used microsomal preparations and an assay similar to ours, but did not include heart failure patients treated with PDE3 inhibitors (Movesian et al., 1991). The other study that reported no difference in enzyme activity between failing and non-failing LVs used a chromatographically isolated soluble PDE3 fraction (von der Leyen et al., 1991). The study reporting decreased activity used a tissue homogenate and a less selective PDE3 inhibitor, and did not include measurement of mRNA abundance (Ding et al., 2005). Whether differences in methodology, starting material or other factors account for these divergent results is unclear.

PDE3 inhibitors are primarily used to treat decompensated heart failure refractory to diuretics and vasodilators (Ding et al., 2005). While they have not been shown to be useful in chronic therapy of heart failure, PDE3 inhibitors are used as a bridge-to-transplant (Lee et al., 1991; Assad-Kottner et al., 2008; Price et al., 2006; Berg et al., 2007) as well as in post-cardiac surgical settings, where their activity is not inhibited by the presence of 3-blockers (Metra et al., 2002). The bridge-to-transplant use may be especially important in severe pediatric heart failure (Price et al., 2006; Berg et al., 2007), where β-adrenergic signaling appears to be different than in adults (Miyamoto et al., 2012) and where ventricular assist devices may not be feasible. Another therapeutic application for PDE3 inhibitors is as antiplatelet/vasodilators in the treatment of peripheral arterial disease (Sallustio et al., 2010). The therapeutic implications of the PDE3A promoter INS/DEL polymorphism are that patients who are INS homozygotes would be predicted to have less tolerance to the pharmacodynamic effects of continuous therapy with any agent that increases cAMP levels including PDE3 inhibitors, while patients who are DEL homozygotes may need an increase in PDE3 inhibitor dose or the addition of a β-agonist (Gilbert et al., 1995) to sustain initial pharmacologic effects. However, increasing the doses of PDE3 inhibitors may lead to a decrease in the selectivity of inhibition and an increased adverse event profile. (Schwinger et al., 1999).

The association of the DEL allele with more pronounced cAMP depletion in LV tissue homogenates is interesting, and could potentially be linked to the statistically greater DEL homozygous genotype frequency in failing vs. non-failing patients. That is, decreased cAMP is a molecular defect in the failing human heart (Feldman et al., 1987; Danielsen et al., 1989; von der Leyen et al., 1991; Bohm et al., 1994), and contributes to decreased PKA-Serine 16 phosphorylation of phospholamban that results in greater inhibition of SERCA2 Schwinger et al., 1999). The tight association of PDE3 inhibitor treatment with up-regulation in PDE3A gene expression and enzyme activity implies that cAMP is elevated in the microdomain(s) where PDE3 inhibitors act, and this elevation is signaled, presumably by translocation of activated PKA, to a nuclear compartment where PDE3A gene transcriptional activity is increased by Serine 133-phosphorylated CREB and its coactivators (Sands and Palmer, 2008). cAMP is freely diffusable within the cell, and intracellular concentrations are regulated locally by PDEs complexed via AKAP scaffolds with PKA regulatory subunits and phosphatases (Mika et al., 2012). This creates a situation whereby cAMP could be elevated by PDE3 inhibitors in the PDE3A containing SERCA2-PLN microdomain (Beca et al., 2013), but decreased elsewhere in the failing cardiac myocyte as a result of increased PDE3A gene expression triggered from the microdomain.

In myocardial tissue the majority of cell volume is provided by cardiac myocytes, and therefore tissue levels of cAMP primarily reflect the cardiac myocyte concentration. In the cardiac myocyte, cAMP concentration is determined by the net effects of generation from adenylyl cyclases and hydrolysis by phosphodiesterases. Decreased cAMP levels in failing ventricular myocardium are due to impaired generation related to desensitization of β-adrenergic signal transduction (Bristow, 2011), which is not offset by less cAMP breakdown from phosphodiesterases. In failing LVs 3-AR density was decreased by 29%, with no difference between genotypes and no trend for a decrease in DEL carriers vs. INS homozygotes. Therefore total β-receptor density, a biologic marker for reduced cAMP generation in failing LVs, was uniformly decreased across PDE inhibitor treatment groups and PDE3A genotypes. In human heart PDE3A is a major PDE, and is the most active enzyme in microsomal preparations influencing cAMP concentrations in microdomains coupled to regulation of contractile function (Movesian et al., 2009; Hambleton et al., 2005; Johnson et al., 2012; Molenaar et al., 2013; Eschenhagen, 2013). In agreement with previous studies (Movesian et al., 1991; Danielsen et al., 1989; von der Leyen et al., 1991). cAMP levels were reduced in homogenates of aliquots of failing left ventricular free wall, in all PDE3A genotypes. Importantly, the degree of cAMP decrease was apparently genetically influenced by the PDE3A INS/DEL polymorphism. That is, compared to INS homozygotes the decrease in cAMP was greater in the two DEL containing genotypes (DEL carriers). Compared to INS homozygotes PDE3A1 mRNA expression and enzyme activity were nonsignificantly (both p values <0.10) increased by ˜30% in DEL carriers regardless of prior treatment with PDE3 inhibitors, which corresponded to a ˜30% decrease in cAMP levels that was statistically significant. For PDE3A enzyme activity and cAMP levels the differences in statistical significance may have been related to the ˜2× greater sample size in the cAMP experiments. Thus it is possible that a selective increase in PDE3A enzyme activity in DEL carriers, either in the SR enriched fraction that the inventors measured or in other PDE3A-PKA containing subcellular compartments where cAMP could be locally increased, led to the genotype-specific further reduction in myocardial cAMP levels. Another possibility for PDE DEL genotype-selective activation of PDE3A gene transcription and expression that is independent of PDE3 inhibitor treatment is Serine 133 phosphorylation of CREB by protein kinases other than PKA (Johannessen et al., 2004). Several of these, such as CaMKII, PKCs, MAPKs and PKD, are up-regulated in the failing human heart. Obviously, more work needs to be done on the relationship of PDE3A genetic variation to regulation of intracellular cAMP levels in the failing heart.

Regardless of the mechanistic basis of DEL genotype-associated effects on myocardial cAMP depletion, this observation suggests that the DEL allele could confer genetic risk for the development of advanced heart failure. The higher frequency of the DEL homozygous genotype in failing LVs, possibly further accentuated in African-Americans, adds support for this hypothesis. Whether the PDE3 DEL allele or the DEL homozygous genotype is a biomarker for the development of advanced heart failure will need to be tested in much larger cohorts.

In conclusion, this study is the first to identify a polymorphic region in the PDE3A promoter that regulates its expression in response to PDE inhibitor treatment and may alter the long term response to PDE3 inhibitors. The identification of a ˜20% subpopulation that is homozygous for the promoter INS and would be expected to develop less tolerance to continuous treatment has the potential to lead to the selection of patients in whom PDE3 inhibition is more likely to have a beneficial effect.

All of the compositions and methods disclosed and claimed herein can be made and executed without undue experimentation in light of the present disclosure. While the compositions and methods of this disclosure have been described in terms of particular embodiments, it will be apparent to those of skill in the art that variations may be applied to the compositions and methods and in the steps or in the sequence of steps of the methods described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VI. REFERENCES

The following references, to the extent that they provide exemplary procedural or other details supplementary to those set forth herein, are specifically incorporated herein by reference.
Assad-Kottner et al., J Card Fail, 14(10):839-843, 2008.
Ausubel et al., Current Protocols in Molecular Biology, John Wiley & Sons, NY, 1989.
Barany, et al., Proc. Natl. Acad. Sci. USA, 88:189-193, 1991.
Beca et al., Circ Res., 112(2):289-297, 2013.
Bellus, J. Macromol. Sci. Pure Appl. Chem., A31(1): 1355-1376, 1994.
Berg et al., J Heart Lung Transplant., 26(5):453-457, 2007.
Bohm et al., Cardiovasc Res., 28(11):1713-1719, 1994.
Bristow M R. Circ Res. 109(10):1176-1194, 2011.
Cohn et al., N Engl J Med. 339(25):1810-1816, 1998.
Danielsen et al., J Cardiovasc Pharmacol., 14(1):171-173, 1989.
de Arruda et al., Expert. Rev. Mol. Diagn., 2:487-496, 2002.
Ding et al., Circulation, 111:2469-2476, 2005.
Durand et al., Ann. Med., 27:311-317, 1995.
Eschenhagen T. Br J Pharmacol., 169(3): 524-527, 2013.
European Appln. 201,184
European Appln. 237,362
European Appln. 258,017
European Appln. 266,032
European Appln. 320 308
European Appln. 329,822
European Appln. 050,424
European Appln. 084,796
Feldman et al., Circulation. 75(2):331-339, 1987.
French Patent 2,650,840
Froehler et al., Nucleic Acids Res., 14(13):5399-5407, 1986.
Frohman, In: PCR Protocols: A Guide To Methods and Applications, Academic Press, N.Y., 1990.
Gilbert et al., Chest., 108(6):1524-1532. 1995.
Great Britain Appln. 2 202 328
Halushka et al., Nat. Genet., 22(3):239-247, 1999.
Hambleton et al., J Biol Chem., 280(47):39168-39174, 2005
Holmes et al., Am Heart J., 110(4):800-806, 1985.
Humphries, et al., In: Molecular Diagnosis of Genetic Diseases, Elles (Ed.), 321-340, 1996.
Inazuka et al., Genome Res, 7(11):1094-1103, 1997.
Innis et al., Proc. Natl. Acad. Sci. USA, 85(24):9436-9440, 1988.
Johannessen et al., Cell Signal. 2004; 16(11):1211-1227.
Johnson et al., Life Sci., 90(9-10):328-336, 2012.
Johnson et al., Nat. Genet., 29(2):233-237, 2001.
Jones, Nature, 199:280-282, 1963.
Ke and Cardon, Bioinformatics, 19(2):287-288, 2003.
Kincaid and Manganiello, Methods Enzymol., 159:457-470, 1988.
Klaassen's The Pharmacological Basis of Therapeutics
Komher, et al., Nucl. Acids. Res. 17:7779-7784, 1989.
Kuppuswamy et al., Proc. Natl. Acad. Sci. USA, 88:1143-1147, 1991.
Kwoh et al., Proc. Natl. Acad. Sci. USA, 86:1173, 1989.
Kwok and Chen, Curr Issues Mol. Biol., 5(2):43-60, 2003.
Kwok et al., Genomics, 23(1):138-144, 1994.
Kwok et al., Genomics, 31(1):123-6, 1996.
Kwok, Annu. Rev. Genomics Hum. Genet., 2:235-258, 2001.
Landegren et al., Science 241:1077-1080, 1988.
Lee et al., J Thorac Cardiovasc Surg., 102(2):246-258, 1991.
Lowes et al., J Cardiac Fail., 11(9):659-669, 2005.
Lowes et al., J Clin Invest., 100(9):2315-2324, 1997.
Lu et al., Biopolymers, 73:606-613, 2004.
Maisel et al., Ann Intern Med., 110(3):195-201, 1989.
Maskin et al., Am J Med., 72(1):113-118, 1982.
Maxam et al., Proc. Natl. Acad. Sci. USA, 74:560, 1977.
Metra et al., J Am Coll Cardiol., 40(7):1248-1258, 2002.
Metra et al., Eur Heart J. 30(24):3015-3026, 2009.
Meyers et al., Science, 230:1242, 1985.
Mika et al., J Mol Cell Cardiol., 52(2):323-329, 2012.
Miyamoto et al., Eur Heart J., July 26. [Epub ahead of print], 2012.
Modrich, Ann. Rev. Genet., 25:229-253, 1991.
Molenaar et al., Br J Pharmacol., 169(3):528-538, 2013.
Movsesian et al., J Clin Invest., 88(1):15-19, 1991.
Movsesian, J. Am. Coll. Cardiol., 34:318-324, 1999.
Mullis et al., Cold Spring Harbor Symp. Quant. Biol. 51:263-273, 1986.
Nickerson et al., Proc. Natl. Acad. Sci. USA, 87:8923-8927, 1990.
Nyren et al., Anal. Biochem. 208:171-175, 1993.
Ohara et al., Proc. Natl. Acad. Sci. USA, 86:5673-5677, 1989.
Orita et al., Genomics, 5:874-879, 1989.
Packer et al., Circulation., 70(6):1038-1047, 1984.
Packer et al., N Engl J Med. 325(21):1468-1475, 1991.
PCT Appln. PCT/US87/00880
PCT Appln. PCT/US89/01025
PCT Appln. WO 88/10315
PCT Appln. WO 89/06700
PCT Appln. WO 93/22456
PCT Appln. WO 95/11995
Physicians Desk Reference
Prezant et al., Hum. Mutat., 1:159-164, 1992.
Price et al., J Card Fail., 12(2):139-143, 2006.
Reinhardt et al., J. Clin. Invest., 95:1528-1538, 1995.
Remington's Pharmaceutical Sciences, 15th Edition, pages 1035-1038 and 1570-1580. 1990.
Ruano et al., Nucl. Acids Res., 17:8392, 1989.
Ruano et al., Nucl. Acids Res., 19:6877-6882, 1991.
Sallustio et al., Curr Vasc Pharmacol., 8(3):363-372, 2010.
Sambrook et al., In: Molecular cloning, Cold Spring Harbor Laboratory Press, Cold Spring Harbor, N.Y., 2001.
Sands and Palmer, Cell Signal., 20(3):460-6, 2008.
Sanger et al., J. Molec. Biol., 94:441, 1975.
Schwinger et al., J Mol Cell Cardiol., 31(3):479-491, 1999.
Shakur et al., Prog. Nucleic Acids Res. Mol. Biol., 66:241-277, 2000.
Sheffield et al., Proc. Natl. Acad. Sci. USA, 86:232-236, 1989.
Simpson et al., Circ Res., 51(6):787-801, 1982.
Singh et al., Basic Res Cardiol., 95(5):389-396, 2009.
Small et al., Methods Enzymol, 343:459-75, 2002.
Smith et al., Biochem Biophys Res Commun, 190(2):516-521, 1993.
Sokolov, Nucl. Acids Res. 18:3671, 1990.
Stevens et al., Biotechniques, 34:198-203, 2003.
Sucharov et al., J Biol Chem., 278(33):31233-31239, 2003.
Sucharov et al., Mol Biol Cell., 19(10):4141-4153, 2008.
Syvanen et al., Genomics 8:684-692, 1990.
Taillon-Miller et al., Genome Res, 8(7):748-754, 1998.
Teerlink et al., Circulation., 101(1):40-46, 2000.
The Merck Index, Eleventh Edition
Turki et al., J. Clin. Invest., 95:1635-1641, 1995.
U.S. Pat. No. 4,582,788
U.S. Pat. No. 4,627,429
U.S. Pat. No. 4,656,127
U.S. Pat. No. 4,659,774
U.S. Pat. No. 4,682,195
U.S. Pat. No. 4,683,194
U.S. Pat. No. 4,683,195
U.S. Pat. No. 4,683,202
U.S. Pat. No. 4,784,857
U.S. Pat. No. 4,800,159
U.S. Pat. No. 4,816,571
U.S. Pat. No. 4,883,750
U.S. Pat. No. 4,946,773
U.S. Pat. No. 4,959,463
U.S. Pat. No. 4,965,188
U.S. Pat. No. 5,126,145
U.S. Pat. No. 5,130,238
U.S. Pat. No. 5,141,813
U.S. Pat. No. 5,169,766
U.S. Pat. No. 5,264,566
U.S. Pat. No. 5,279,721
U.S. Pat. No. 5,428,148
U.S. Pat. No. 5,554,744
U.S. Pat. No. 5,574,146
U.S. Pat. No. 5,602,244
U.S. Pat. No. 5,605,798
U.S. Pat. No. 5,645,897
U.S. Pat. No. 5,662,925
U.S. Pat. No. 5,705,629
U.S. Pat. No. 5,788,983
U.S. Pat. No. 5,840,873
U.S. Pat. No. 5,843,640
U.S. Pat. No. 5,843,650
U.S. Pat. No. 5,843,651
U.S. Pat. No. 5,843,663
U.S. Pat. No. 5,846,708
U.S. Pat. No. 5,846,709
U.S. Pat. No. 5,846,717
U.S. Pat. No. 5,846,726
U.S. Pat. No. 5,846,729
U.S. Pat. No. 5,846,783
U.S. Pat. No. 5,849,481
U.S. Pat. No. 5,849,483
U.S. Pat. No. 5,849,486
U.S. Pat. No. 5,849,487
U.S. Pat. No. 5,849,497
U.S. Pat. No. 5,849,546
U.S. Pat. No. 5,849,547
U.S. Pat. No. 5,851,770
U.S. Pat. No. 5,851,772
U.S. Pat. No. 5,853,990
U.S. Pat. No. 5,853,992
U.S. Pat. No. 5,853,993
U.S. Pat. No. 5,856,092
U.S. Pat. No. 5,858,652
U.S. Pat. No. 5,861,244
U.S. Pat. No. 5,863,732
U.S. Pat. No. 5,863,753
U.S. Pat. No. 5,866,331
U.S. Pat. No. 5,866,337
U.S. Pat. No. 5,866,366
U.S. Pat. No. 5,900,481
U.S. Pat. No. 5,905,024
U.S. Pat. No. 5,910,407
U.S. Pat. No. 5,912,124
U.S. Pat. No. 5,912,145
U.S. Pat. No. 5,912,148
U.S. Pat. No. 5,916,776
U.S. Pat. No. 5,916,779
U.S. Pat. No. 5,919,626
U.S. Pat. No. 5,919,630
U.S. Pat. No. 5,922,574
U.S. Pat. No. 5,925,517
U.S. Pat. No. 5,925,525
U.S. Pat. No. 5,928,862
U.S. Pat. No. 5,928,869
U.S. Pat. No. 5,928,870
U.S. Pat. No. 5,928,905
U.S. Pat. No. 5,928,906
U.S. Pat. No. 5,929,227
U.S. Pat. No. 5,932,413
U.S. Pat. No. 5,932,451
U.S. Pat. No. 5,935,791
U.S. Pat. No. 5,935,825
U.S. Pat. No. 5,939,291
U.S. Pat. No. 5,942,391
U.S. Pat. No. 5,952,174
U.S. Pat. No. 6,113,940
Ugozzoll et al., GATA 9:107-112, 1992.
von der Leyen et al., Naunyn Schmiedebergs Arch Pharmacol., 344(1):90-100, 1991.
Walker et al., Proc. Natl. Acad. Sci. USA, 89:392-396, 1992.
Wang and Dhalla, Mol. Cell Biochem., 214:131-155, 2000.
Wartell et al., Nucl. Acids Res., 18:2699-2706, 1990.
Waspe et al., J Clin Invest., 85(4):1206-1214, 1990.
Wechsler et al., J Biol Chem., 277(41):38072-38078, 2002.
Winter et al., Proc. Natl. Acad. Sci. USA, 82:7575, 1985.

What is claimed:

1. A method of prophylactically treating heart failure comprising treating a subject after the subject is determined to exhibit a −1130 to −1159 deletion polymorphism in the promoter of a phosphodiesterase type 3A (PDE3A) gene, wherein said treating does not comprise a PDE3A inhibitor.

2. The method of claim 1, wherein treating comprises a beta blocker.

3. The method of claim 1, wherein treating comprises an agent that prevents reductions in intracellular cAMP.

4. The method of claim 1, wherein said subject is heterozygous for the deletion polymorphism.

5. The method of claim 1, wherein said subject is homozygous for the deletion polymorphism.

6. The method of claim 1, wherein determining comprises nucleic acid amplification, nucleic acid hybridization, restriction fragment length polymorphism (RFLP) analysis, single stranded conformational polymorphism (SSCP) analysis, nucleic acid sequencing, denaturing high performance liquid chromatography, allele specific amplification, allele specific hybridization, comparative genome hybridization, and/or Southern blotting.

7. The method of claim 6, wherein nucleic acid amplification comprises polymerase chain reaction amplification or ligase chain reaction amplification.

8. The method of claim 6, wherein nucleic acid hybridization detection method comprises an allele specific oligonucleotide probe or a microarray of nucleic acid probes.

9. The method of claim 1, wherein said subject has been diagnosed with one or more of hypertension, myocardial infarction, atherosclerosis, cardiac hypertrophy, left ventricular remodeling, or coronary artery disease.

10. The method of claim 1, wherein said subject is a carrier of a gene known to cause cardiomyopathy.

11. A method for predicting development of heart failure in a subject not suffering from heart failure comprising analyzing a biological sample from the subject for the presence of a −1130 to −1159 insertion/deletion polymorphism in the promoter of one or more phosphodiesterase type 3A (PDE3A) genes of the subject, wherein a subject determined to be homozygous or heterozygous for the deletion polymorphism is at increased risk of developing heart failure as compared to an individual who is homozygous for the insertion polymorphism.

12. The method of claim 11, wherein said subject is heterozygous for the deletion polymorphism.

13. The method of claim 11, wherein said subject is homozygous for the deletion polymorphism.

14. The method of claim 11, further comprising preparing a report containing information regarding the genotype of one or more PDE3A genes of the subject.

15. The method of claim 11, wherein determining comprises nucleic acid amplification, nucleic acid hybridization, restriction fragment length polymorphism (RFLP) analysis, single-stranded conformational polymorphism (SSCP) analysis, nucleic acid sequencing, denaturing high performance liquid chromatography, allele specific amplification, allele specific hybridization, comparative genome hybridization, and/or Southern blotting.

16. The method of claim 15, wherein nucleic acid amplification comprises polymerase chain reaction amplification or ligase chain reaction amplification.

17. The method of claim 15, wherein nucleic acid hybridization detection method comprises an allele specific oligonucleotide probe or a microarray of nucleic acid probes.

18. The method of claim 11, wherein said subject has been diagnosed with one or more of hypertension, myocardial infarction, atherosclerosis, cardiac hypertrophy, left ventricular remodeling, or coronary artery disease.

19. The method of claim 11, further comprising scheduling said subject for one or more follow up diagnostic procedures selected from measurement of ventricular volume, measurement of ejection fraction, and/or surveillance for progressive remodeling.

20. The method of claim 11, wherein said subject is a carrier of a gene known to cause cardiomyopathy.

21. A method for treating early stage heart failure in a subject comprising treating a subject after the subject is determined to exhibit a −1130 to −1159 deletion polymorphism in the promoter of phosphodiesterase type 3A (PDE3A) gene, wherein said treating does not comprise a PDE3A inhibitor.

22. The method of claim 21, wherein said subject is heterozygous for the deletion polymorphism.

23. The method of claim 21, wherein said subject is homozygous for the deletion polymorphism.

24. The method of claim 21, wherein treating comprises a beta blocker.

25. The method of claim 21, wherein treating comprises an agent that prevents reductions in intracellular cAMP.

26. The method of claim 21, wherein determining comprises nucleic acid amplification, nucleic acid hybridization, restriction fragment length polymorphism (RFLP) analysis, single-stranded conformational polymorphism (SSCP) analysis, nucleic acid sequencing, denaturing high performance liquid chromatography, allele specific amplification, allele specific hybridization, comparative genome hybridization, and/or Southern blotting.

27. The method of claim 26, wherein nucleic acid amplification comprises polymerase chain reaction amplification or ligase chain reaction amplification.

28. The method of claim 26, wherein nucleic acid hybridization detection method comprises an allele specific oligonucleotide probe or a microarray of nucleic acid probes.

29. The method of claim 21, wherein said subject has been diagnosed with one or more of hypertension, myocardial infarction, atherosclerosis, cardiac hypertrophy, left ventricular remodeling or coronary artery disease.

30. The method of claim 21, wherein said subject is a carrier of a gene known to cause cardiomyopathy.


SEQUENCE LISTING (1 sequence)

SEQ ID NO: 1 - DNA, 29 bp, Artificial
  Synthetic oligonucleotide
ttctcatatctacttatgtcataatatta